(19) United States
(12) Patent Application Publication
(10) Pub. No.: US 2024/0216403 A1
(43) Pub. Date: Jul. 4, 2024

(54) METHODS AND COMPOSITIONS FOR INHIBITING FIBROBLAST ACTIVATION

(71) Applicant: NOVOCURE GMBH, Root D4 (CH)

(72) Inventors: Lilach Avigdor, Haifa (IL); Anat Klein-Goldberg, Haifa (IL); Tali Voloshin-Sela, Haifa (IL); Rom Paz, Haifa (IL)

(21) Appl. No.: 18/398,557

(22) Filed: Dec. 28, 2023

Related U.S. Application Data
(60) Provisional application No. 63/477,565, filed on Dec. 29, 2022.

(51) Int. Cl. A61K 31/69; A61K 31/277; A61K 31/366; A61K 31/395; A61K 31/404; A61K 31/454; A61K 31/46; A61K 31/4709; A61K 31/497; A61K 31/5377; A61K 47/68; A61K 51/08; A61N 1/36; A61P 35/00

(52) U.S. Cl. CPC A61K 31/69; A61K 31/277; A61K 31/366; A61K 31/395; A61K 31/404; A61K 31/454; A61K 31/46; A61K 31/4709; A61K 31/497; A61K 31/5377; A61K 47/68031; A61K 47/6849; A61K 51/088; A61N 1/36002; A61P 35/00

(57) ABSTRACT

Disclosed are methods of treating a subject in need thereof comprising applying an alternating electric field, to a target site of the subject in need thereof; and administering a cancer associated fibroblast (CAF) inhibitor to the subject in need thereof. Disclosed are methods of reducing or preventing CAF activation comprising applying alternating electric fields, to a population of cells comprising one or more fibroblasts; and contacting a CAF activation inhibitor to the population of cells. Disclosed are methods of reducing CAF signaling comprising applying alternating electric fields, to a population of cells comprising one or more fibroblasts; and contacting a cancer associated fibroblast (CAF) signaling inhibitor to the population of cells.

### CROSS-REFERENCE TO RELATED APPLICATIONS

This application claims the benefit of U.S. Provisional Patent Application No. 63/477,565, filed Dec. 29, 2023, which is incorporated by reference herein in its entirety.

### BACKGROUND

Fibroblasts are responsible for wound healing. They contract the wound and produce extra cellular matrix (ECM) proteins such as collagen, elastin and other enzymes and cytokines in order to repair damaged tissue. Unlike in normal wound healing that is eventually resolved, cancer cells influence the stroma of the tumor to continue and secret inflammatory cytokines and increased ECM proteins without reaching homeostasis. Cancer activated fibroblasts (CAFs) are fibroblast populations that reside within the tumor tissue expressing and secreting proteins and by doing so they influence remodeling of the tumor. CAFs usually express FAP (fibroblast activation protein), Alpha smooth muscle actin (aSMA), Platelet derived growth factor receptor beta (PDGFRb), integrin beta 1(ITDB1), CD26 and caveolin-1 (CAV1).

CAFs not only produce and secrete ECM proteins, they also actively participate in the ECM proteolysis, crosslinking and assembly processes of ECM proteins. In such a rigid and highly cross-linked tumor stroma, drug penetration is one potential reason for tumor cells to escape therapy. In addition, CAF-mediated ECM remodeling is a highly responsive process of receiving, processing and responding to the cellular, molecular and mechanical signals in the tumor microenvironment.

What is needed are alternative strategies to target cancer activated fibroblasts and to treat cancers.

### BRIEF SUMMARY

Disclosed herein are studies that TTFields increase activation of fibroblasts in vivo and in vitro. Therefore, treatment with TTFields while inhibiting the activation of cancer associated fibroblast would result in better patient outcome.

Disclosed are methods of treating a subject in need thereof comprising applying alternating electric fields, to a target site of the subject in need thereof; and administering a cancer associated fibroblast (CAF) inhibitor to the subject in need thereof. In some aspects, a CAF inhibitor can be a CAF activation inhibitor or a CAF signaling inhibitor.

Disclosed are methods of treating a subject in need thereof comprising applying alternating electric fields, to a target site of the subject in need thereof; and administering a CAF activation inhibitor to the subject in need thereof.

Disclosed are methods of treating a subject in need thereof comprising applying alternating electric fields, to a target site of the subject in need thereof; and administering a CAF signaling inhibitor to the subject in need thereof.

Disclosed are methods of reducing or preventing CAF activation comprising applying alternating electric fields, to a population of cells comprising one or more fibroblasts; and contacting a CAF activation inhibitor to the population of cells.

Disclosed are methods of reducing CAF signaling comprising applying alternating electric fields, to a population of cells comprising one or more fibroblasts; and contacting a cancer associated fibroblast (CAF) signaling inhibitor to the population of cells.

Disclosed are methods of decreasing tumor growth in a subject in need thereof comprising applying alternating electric fields, to a target site of the subject in need thereof; and administering a CAF inhibitor to the subject in need thereof.

Disclosed are methods of decreasing tumor invasion in a subject in need thereof comprising applying alternating electric fields, to a target site of the subject in need thereof; and administering a CAF inhibitor to the subject in need thereof.

Disclosed are methods of decreasing tumor angiogenesis in a subject in need thereof comprising applying alternating electric fields, to a target site of the subject in need thereof; and administering a CAF inhibitor to the subject in need thereof.

Disclosed are methods of decreasing tumor metastasis in a subject in need thereof comprising applying alternating electric fields, to a target site of the subject in need thereof; and administering a CAF inhibitor to the subject in need thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIGS. 1A-1C show the up regulation of PDGFRb in response to TTFields.

FIGS. 2A-2C show the up regulation of aSMA in response to TTFields.

FIG. 3 shows in detail the results presented in FIG. 2C where not only the amount of cells expressing aSMA is increased but also the mean fluorescent intensity (MFI) is increased per cell indicating that cells express more aSMA.

FIG. 4A shows representative confocal images of aSMA expression in fibroblasts following 48 hours of TTFields application, FIG. 4B shows an increase in aSMA expression is observed in fibroblasts following 48 hours of TTFields application compared to untreated fibroblasts.

FIGS. 5A and 5B show aSMA expression is increased in fibroblasts following incubation with conditioned medium (CM) derived from TTFields treated cancer cells. FIG. 5A shows a schematic of the experiments. FIG. 5B shows FACS analysis graph and mean fluorescence of aSMA.

FIG. 6 shows the process of isolation and characterization of fibroblasts from cells derived from single cell suspended tumors in lungs of LLC2 bearing mice treated with TTFields application or heat sham as control.

FIG. 7 shows a scheme of FACS analysis gating strategy to distinguish activated fibroblasts in samples of cells derived from tumor single cell dissociation

FIG. 8 shows aSMA expression is increased n CD45-/CD31-/PDGFR+CD29+ cells derived from tumors following TTFields application.

FIG. 9 shows aSMA expression is increased n CD45-/CD31-/PDGFR+CD29+ cells derived from tumors following TTFields application.

FIG. 10 shows secretion of CAF markers, such as MCP1 and CD26, indicating differentiation of fibroblasts to CAFs in TTFields treated cells. Conditioned media derived from MRC5 fibroblasts following TTFields application for 48 or 72 hours at 150 kHz, incubated with a secretory cytokine array assay according to product manual, revealed an increase in secretion of MCP1 (monocyte chemoattractant protein 1) that is secreted by activated fibroblasts and CD26, which is a CAF marker indicating differentiation of fibroblast to CAFs. CD26 was increased by 1.5 and 2 fold following TTFields treatment for 48 and 72 hours, respectively and MCP-1 was increased by 1.5 and 1.4 fold following TTFields treatment for 48 and 72 hours, respectively. Analysis was performed by HLImage++ quickspot software.

FIG. 11 shows an example of the concentration (pg/ml) of active TGF-β1 and total TFG-β1 measured in the serum of mice injected with LL/2 NSCLC cells and treated with TTFields or sham heat for 8 days using ELISA. (N control=7 TTFields=7)

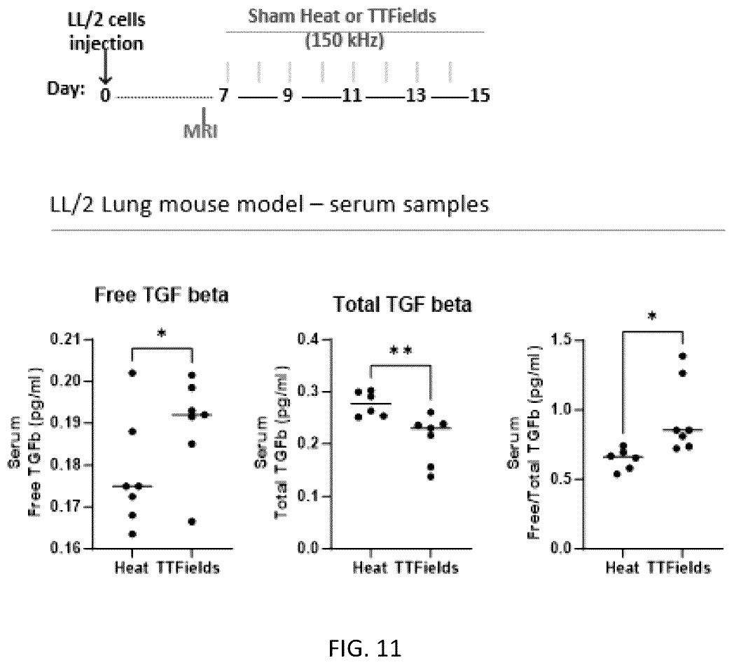

FIG. 12 shows an example of the concentration (pg/mg) of active TGF-β1 and total TFG-β1 measured in the lung tumors of mice injected with LL/2 NSCLC cells and treated with TTFields or sham heat for 8 days using ELISA. (N control=7 TTFields=7).

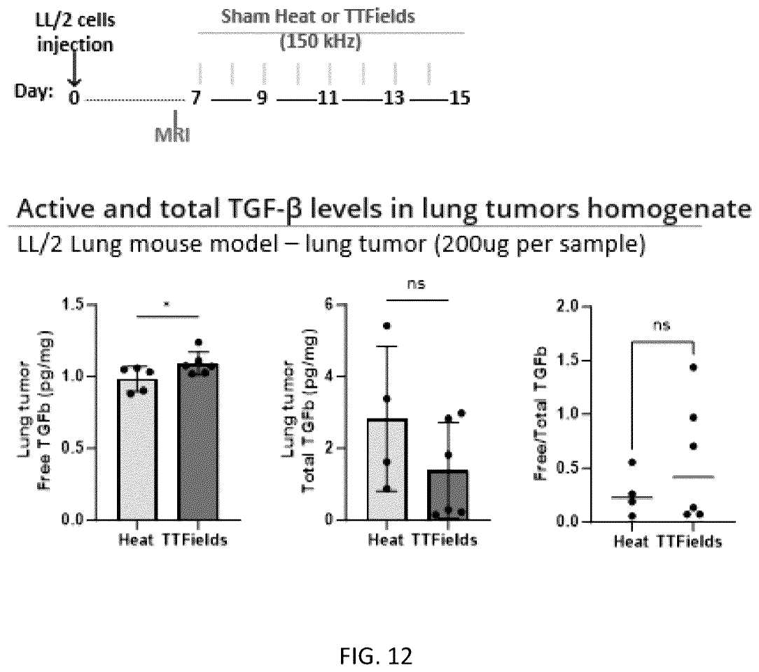

FIG. 13 shows tissue expression of Collagen 1 and Lox. TTFields were continuously applied for 10 days in a frequency of 200 kHz to ovarian tumor bearing mice. Treatment with TTFields significantly elevated LOX protein expression.

FIG. 14 shows tissue expression of Collagen 4 and Fibronectin. TTFields were continuously applied for 10 days in a frequency of 200 kHz to ovarian tumor bearing mice. Treatment with TTFields significantly elevated Collagen 4 protein expression.

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a CAF activation inhibitor” includes a plurality of such CAF activation inhibitors, reference to “the cell” is a reference to one or more cells and equivalents thereof known to those skilled in the art, and so forth.

As used herein, a “target site” is a specific site or location within or present on a subject or patient. For example, a “target site” can refer to, but is not limited to a cell (e.g., a cancer cell or a cancer associated fibroblast), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain. In some aspects, a cell or population of cells that can be a target site or a target cell include, but are not limited to, a cancer cell (e.g., a lung cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical field delivered to a subject, a sample obtained from a subject or to a specific location within a subject or patient (e.g., a target site such as a cell). In some aspects, the alternating electrical field can be in a single direction or multiple directional, e.g., alternate directions across the target site. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields.

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. “Subject” can be used interchangeably with “individual” or “patient.” For example, the subject of administration can mean the recipient of the alternating electrical field. For example, the subject of administration can be a subject with cancer, e.g., ovarian cancer or lung cancer.

By “treat” is meant to administer or apply a therapeutic, such as alternating electric fields and a cancer associated fibroblast (CAF) inhibitor, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having lung cancer can comprise delivering a therapeutic to a cell in the subject.

By “prevent” is meant to minimize or decrease the chance that a subject develops cancer.

As used herein, the terms “administering” and “administration” refer to any method of providing a CAF inhibitor to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise applying alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 150 and 300 kHz, between 180 and 220 kHz, or between 210 and 400 kHz. In some aspects, the frequency of the alternating electric fields can be 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric field can be between 0.5 and 4 V/cm RMS. In some aspects, the field strength of the alternating electric field can be between 1 and 4 V/cm RMS. In some aspects, different field strengths can be used (e.g., between 0.1 and 10 V/cm RMS). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric field can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric field can be applied every day for a two hour duration. For example, the alternating electric field can be applied for at least 4 hours per day, at least 8 hours per day, at least 12 hours per day, at least 16 hours per day, or at least 20 hours per day. In some aspects the alternating electric field can be applied for at least 4, 8, 12, 16, or 20 hours per day for at least 2 days. In some aspects the alternating electric field can be applied for at least 4, 8, 12, 16, or 20 hours per day for at least 3 days. In some aspects the alternating electric fields can be applied for at least 4, 8, 12, 16, or 20 hours per day for at least 7 days.

In some aspects, the consecutive exposure may last for at least 6 hours, at least 12 hours, at least 24 hours, at least 36 hours, at least 48 hours, or at least 72 hours or more.

In some aspects, the cumulative exposure may last for at least 42 hours, at least 84 hours, at least 168 hours, at least 250 hours, at least 400 hours, at least 500 hours, at least 750 hours, or more.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell. C. Methods of Treating

Disclosed are methods of treating a subject in need thereof comprising applying an alternating electric field, to a target site of the subject in need thereof; and administering a cancer associated fibroblast (CAF) inhibitor to the subject in need thereof. In some aspects, a CAF inhibitor can be a CAF activation inhibitor or a CAF signaling inhibitor. Thus, in some aspects, a CAF inhibitor can prevent or reduce CAF activation or can prevent or reduce signaling from CAFs.

Disclosed are methods of treating a subject in need thereof comprising applying alternating electric fields, to a target site of the subject in need thereof; and administering a CAF activation inhibitor to the subject in need thereof.

Fibroblast activation protein (FAP) is a cell membrane-bound serine peptidase, overexpressed in cancer-associated fibroblasts and activated fibroblasts at wound healing/inflammatory sites.

Platelet-derived growth factors (PDGFs) and their tyrosine kinase receptors (PDGFRs) have been implicated in the pathogenesis of a number of tumor types and play an important role in angiogenesis. Tumor growth can be promoted by PDGF via autocrine stimulation of malignant cells, by overexpression or over activation of PDGFRs, or by stimulation of angiogenesis within the tumor.

Hedgehog pathway inhibitors have been tested clinically to reduce CAF activation: Hedgehog signaling intersects with CAF heterogeneity in pancreatic ductal adenocarcinoma (PDAC). Acute inhibition of this pathway can change the CAF activation ratio, transforming the tumor microenvironment into a more immunosuppressive phenotype.

Hyaluronic acid depletion agents—Hyaluronic acid is a major component of the ECM that has been shown to bind to tumor cells contributing to the migration and invasion of CAF-guided tumor cells. Angiotensin inhibition also has been encouraging in clinical trials in attenuation of collagen and hyaluronan deposition by CAFs.

IL-1R inhibition via inhibitors such as anakinra is in clinical studies since it is known that pancreatic tumor-secreted IL-1 activated the LIF/JAK/STAT pathway to activate inflammatory CAFs (iCAFs) in PDAC to promote tumor growth.

Hsp90 (heat shock protein 90) is a chaperone protein that assists other proteins to fold properly, stabilizes proteins against heat stress, and aids in protein degradation. It also stabilizes a number of proteins required for tumor growth. In some aspects, Hsp90 inhibitors can limit activation of cancer associated fibroblasts (CAF).

Leucine rich repeat containing 15 (LLRC15) is a cell membrane expressed protein that in humans is encoded by the LRRC15 gene, which is involved in cell-cell and cell-ECM interactions. LRRC15 lacks obvious intracellular domains. LRRC15 displays a highly restricted expression pattern, but is expressed in areas that make up innate immune barriers such as the placenta, skin, activated fibroblasts in wounds, and lymphoid tissues such as the spleen. LRRC15 may play some role in innate immunity. LRRC15 is aberrantly expressed in cancer. It is highly expressed in CAFs within the stroma of numerous solid tumors and directly expressed in mesenchymal tumors such as glioblastoma, sarcomas, and melanoma.

In some aspects, a CAF activation inhibitor can be, but is not limited to, a fibroblast activation protein (FAP) inhibitor, a selective platelet derived growth factor receptor beta (PDGFRb) inhibitor, a HSP90 inhibitor, LLRC15 inhibitor, Hedgehog inhibitor, Hyaluronic acid depletor or IL-1R inhibitor.

In some aspects, a FAP inhibitor can be, but is not limited to, small molecule inhibitors such as Talabostat or FAP-2286, Simlukafusp alfa (FAP-IL2v, R06874281/RG7461), SP-12786, Gemigliptin, FAPI-4, FAPI-2, OncoFAP, FAPI-34, TAM558, BR102910, or a neutralizing antibody against FAP such as TAM558. In some aspects, the FAP inhibitor can be one or more of the inhibitors provided in www.medchemexpress.com/search.html?q=FAP+ inhibitor&ft=&fa=&fp=&fsp=&ftag=&fsc=, which is incorporated herein by reference for its teaching of FAP inhibitors.

In some aspects, a selective PDGFRb inhibitor refers to an inhibitor that only binds to PDGFRb and does not bind to other PDGFR ligands. In some aspects, a selective PDGFRb inhibitor can be, but is not limited to, sPDGFRbIg, Crenolanib, Orantinib, Linifanib, CP-673451, SU16f, Tyrphostin AG 1296, Tyrphostin AG1433, SU4312, SU6668, AC710, JNJ 10198409, DMPQ, PD 166285, KG5, PDGFR tyrosine kinase inhibitor III, PDGFR-IN-1, Ansornitinib, PP58, GZD856, or Seralutinib.

In some aspects, a Hsp90 inhibitor can be, but is not limited to, XL888, Geldanamycin, Gedunin, Tanespimycin, Luminespib, Radicicol, 17-DMAG, EC144, Herbimycin A, Ganetespib, Onalespib, NVP-BEP800, SNX-2112, PF-04929113, KW-2478, NMS-E973, Zelavespib, Pseudolaric acid A, VER-49009, Pimitespib, CH538303, VER-50589, Cucurnitacin, HSP990, BIIV 021, CCT 018159,17-AAG. In some aspects, the Hsp90 inhibitor can be one or more of the inhibitors provided in www.medchemexpress.com/search.html?q=hsp90&ft=&fa=&fp=&fsp=&ftag=&fsc=, which is incorporated herein by reference for its teaching of Hsp90 inhibitors.

In some aspects, a LLRC15 inhibitor can be, but is not limited to, ABBV-085.

In some aspects, a Hedgehog inhibitor can be, but is not limited to IPI-926, Vismodegib, KAAD-Cyclopamine, Cyclopamine, Robotnikinin, 3-epi-Vitamin D3, Sonidegib (NVP-LDE225), Jervine, HPI 1, Dynarrestin, TAK-441, TPB15, JK184, Ciliobrevin A, Hh-Agl1.5, SANT 2, MK-4101, MRT 10, U 18666A, CUR61414 or Itraconazole (R51211). In some aspects, the Hedgehog inhibitor can be one or more of those provided in www.medchemexpress.com/search.html?q=hedgehog&ft=&fa=&fp=&fsp=&ftag=&fsc, which is incorporated herein by reference for its teaching of Hedgehog inhibitors.

In some aspects, a hyaluronic acid depletion agent can be, but is not limited to PEGPH20, 4-MU, Hyaluronidase or an angiotensin receptor agent that attenuates Hyaluronan secretion such as Losartan, Mopivabil, Pratosartan, Mepixetil, LY285434, Azilsartan mopivanil, Tasosartan, ZD 7155, BIBS 39 or CGP 48369. In some aspects, the hyaluronic acid depletion agent can be one or more of the inhibitors those provided in www.tocris.com/search?keywords=angiotensin+ inhibitor or www.medchemexpress.com/search.html?q=angiotensin+II+receptor+antagonist&ft=&fa=&fp=&fsp=&ftag=&fsc=, which is incorporated herein by reference for its teaching of hyaluronic acid depletion.

In some aspects, an IL-1R inhibitor can be, but is not limited to Anakinra, AF12198 or Diacerein.

Disclosed are methods of treating a subject in need thereof comprising applying an alternating electric field, to a target site of the subject in need thereof; and administering a CAF signaling inhibitor to the subject in need thereof.

In some aspects, a CAF signaling inhibitor can be, but is not limited to, a Lox (lysyl oxidase) inhibitor or LoxL (lysyl oxidase like) 1-4 inhibitor. In some aspects, LoxL inhibitors can be, but are not limited to, monoclonal antibodies against Lysyl oxidase like-2/3 such as simtuzumab, PXS-5120A, PAT-1251, PXS-4787, PXS-6302, anti-LOXL 1-4 antibody or a small molecule inhibitor such as PXS-5153A. In some aspects, a LOX inhibitor can be, but is not limited to, LOX-IN-3, β-Aminopropionitrile (BAPN), CCT365623, PXS-6302, PXS-5505, mIR142-3p or PXS-4787. In some aspects, the LOX inhibitor can be one or more of the inhibitors provided in www.medchemexpress.com/search.html?q=Lysyl+oxidase&ft=&fa=&fp=&fsp=&ftag=&fsc=& type=inhibitors-and-agonists, which is incorporated herein by reference for its teaching of LOX inhibitors.

In some aspects, a CAF signaling inhibitor can be, but is not limited to, a CXCR4 inhibitor or a CXCL12 inhibitor. CXCL12-CXCR4 axis antagonism has been tested in clinical trials since CXCL12 is secreted by CAFs and promotes tumorigenesis. CXCR4 inhibitors include, for example, Olaptesed (NOX-A12), BL-8040 CXCR4 Antagonist, TC14012, KRH 2955, ITlt, Plerixafor (AMD 3100), WZ811, USL311, LY2510924, MSX-127, MSX-130, AMD 3465, CTCE 9908, Tannic acid, Motixafortide (BL-8040), MSX-122, Balixafortide (P0L6326), ATI 2341cr CXCR4 antibody. CXCL12 inhibitors include, for example, UNBS5162, LIT-927, or CXCL12 antibody. In some aspects, any of the CAF signaling inhibitors described in Izumi et al. Int J Cancer. 2016 Mar. 1; 138(5):1207-19, incorporated herein by reference in its entirety.

In some aspects, the target site comprises one or more cancer cells. In some aspects, the target site comprises one or more mesothelioma cells, ovarian cancer cells, cervical cancer cells, lung cancer cells, brain cancer cells, pancreatic cancer cells, breast cancer cells, hepatocellular cancer cells, renal cancer cells or colon cancer cells. In some aspects, the target site comprises cancer cells from any type of cancer.

In some aspects, the alternating electric field is applied before, after, or simultaneously with administering the CAF inhibitor (e.g., a CAF activation inhibitor or CAF signaling inhibitor). In some aspects, the step of applying the alternating electric field begins at least one hour before a CAF inhibitor (e.g., a CAF activation inhibitor or CAF signaling inhibitor) is administered. In some aspects, the step of applying the alternating electric fields begins at least 30 minutes before a CAF inhibitor (e.g., a CAF activation inhibitor or CAF signaling inhibitor) is administered. In some aspects, applying the alternating electric field simultaneously can mean applying within 5, 10, 15, 20, 25, 30, 35, 40, 45, 50, 55, or 60 minutes before or after administering a CAF inhibitor (e.g., a CAF activation inhibitor or CAF signaling inhibitor). In some aspects, the alternating electric field can be applied and the CAF inhibitor (e.g., a CAF activation inhibitor or CAF signaling inhibitor) administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the CAF inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the effects of the CAF activation inhibitor can be determined by counting the number of activated fibroblasts and/or detecting the presence or absence of CAF markers. For example, in some aspects, in some aspects, the CAF activation inhibitor decreases the number of activated fibroblasts. In some aspects, the CAF activation inhibitor inhibits or decreases FAP, alpha smooth muscle actin (aSMA), or PDGFRb expression, Hedgehog expression, or hyaluronic acid formation.

In some aspects, the effects of the CAF signaling inhibitors can be determined by detecting downstream signaling events. In some aspects, the CAF signaling inhibitors can block ligands or receptors involved in CAF signaling thereby preventing a signaling event.

In some aspects, the CAF inhibitor blocks upregulation of FAP, aSMA, PDGFRb, and/or Hedgehog expression and/or hyaluronic acid formation caused by alternating electric fields. In some aspects, the CAF inhibitor blocks signaling through FAP, aSMA, PDGFRb, and/or Hedgehog caused by alternating electric fields.

In some aspects, the CAF inhibitor prevents PDGFRb from interacting with or binding to PDGF. For example, in some aspects, the CAF inhibitor binds to or interacts with PDGFRb thus preventing PDGFRb from interacting with or binding to its ligand, PDGF.

In some aspects, the CAF activation inhibitor prevents Hsp90 ATPase activity. In some aspects, Hsp90 ATPase activity upregulates fibroblast activation therefore preventing or inhibiting Hsp90 ATPase activity can inhibit CAF activation.

In some aspects, extracellular Hsp90a interacts with ER stress (which can be caused by alternating electric fields) to promote fibroblasts activation. Therefore, in some aspects, an Hsp90 inhibitor can prevent or inhibit CAF activation.

In some aspects, Hedgehog pathway intersects with CAF heterogeneity to promote fibroblasts activation. Therefore, in some aspects, a Hedgehog inhibitor can prevent or inhibit CAF activation.

In some aspects, inhibition of hyaluronic acid formation inhibits binding of hyaluronic acid to tumor cells thereby inhibiting the encouragement of CAFs to migrate and invade the tumor and promote tumorigenesis.

In some aspects, inhibition of angiotensin inhibits hyaluronic acid formation which inhibits binding of hyaluronic acid to tumor cells thereby encouraging CAFs to migrate and invade the tumor and promote tumorigenesis.

In some aspects, inhibition of IL-1R inhibits binding to IL-1R and inhibits signal transduction that leads to activation of CAFs.

In some aspects, the CAF signaling inhibitor prevents Lox or Loxl from covalently cross-linking collagen and elastin in extracellular matrix (ECM). The lysyl oxidase (LOX) family of enzymes plays a critical role in the formation, maturation, and remodeling of extracellular matrix (ECM) which supports tumor growth and metastasis. Therefore, in some aspects, inhibiting signaling via Lox or Loxl can treat cancers.

In some aspects, inhibition of CXCR4 inhibits binding of CXCL12 to CXCR4 and this in turn inhibits secretion of CXCL12 from CAFs, which promotes tumorigenesis.

In some aspects, the frequency of the alternating electric field is between 100 and 500 kHz. In some aspects, the frequency of the alternating electric fields is between 50 kHz and 1 MHz. In some aspects, the frequency of the alternating electric field is about 150 kHz, about 200 kHz, or about 250 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 150 and 300 kHz, between 180 and 220 kHz, or between 210 and 400 kHz. In some aspects, the frequency of the alternating electric fields can be 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the field strength can be about or at least 1.75 V/cm RMS. In some embodiments the field strength is about or at least 1 V/cm RMS. In some aspects, the alternating electric field has a field strength of about or at least 0.9 V/cm RMS. In some aspects, the alternating electric field has a field strength of any of those described herein. In other embodiments, combinations of field strengths are applied, for example combining two or more frequencies at the same time, and/or applying two or more frequencies at different times.

In some aspects, the alternating electric field can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric field can be applied every day for a two hour duration. For example, the alternating electric field can be applied for at least 4 hours per day, at least 8 hours per day, at least 12 hours per day, at least 16 hours per day, or at least 20 hours per day. In some aspects the alternating electric field can be applied for at least 4, 8, 12, 16, or 20 hours per day for at least 2 days. In some aspects the alternating electric field can be applied for at least 4, 8, 12, 16, or 20 hours per day for at least 3 days. In some aspects the alternating electric fields can be applied for at least 4, 8, 12, 16, or 20 hours per day for at least 7 days.

In some aspects, the consecutive exposure may last for at least 6 hours, at least 12 hours, at least 24 hours, at least 36 hours, at least 48 hours, or at least 72 hours or more.

In some aspects, the cumulative exposure may last for at least 42 hours, at least 84 hours, at least 168 hours, at least 250 hours, at least 400 hours, at least 500 hours, at least 750 hours, or more.

In some aspects, the disclosed methods of treating can further comprise administering a cancer therapeutic. In some aspects, the cancer therapeutic is a known cancer therapeutic other than the CAF activation inhibitor or CAF signaling inhibitor. For example, the cancer therapeutic can be, but is not limited to, chemotherapy, radiation, immunotherapy, or hormone therapy. In some aspects, the cancer therapeutic can be, but is not limited to, tyrosine kinase inhibitors, PI3K inhibitors, Akt inhibitors, VEGF inhibitors, or FGF inhibitors.

In some aspects, the alternating electric fields are applied before, after, or simultaneously with administering the cancer therapeutic. In some aspects, the CAF inhibitor (e.g., CAF activation inhibitor or CAF signaling inhibitor) is applied before, after, or simultaneously with administering the cancer therapeutic. In some aspects, the CAF inhibitor (e.g., CAF activation inhibitor or CAF signaling inhibitor) and cancer therapeutic are administered simultaneously and the alternating electric fields are applied before or after the CAF activation inhibitor or CAF signaling inhibitor and cancer therapeutic.

In some aspects, after applying alternating electric fields and prior to administering a CAF inhibitor, the method can further comprise a step of detecting an increase in aSMA, FAP, PDGFRO, ITDB1, CAV1, and/or CD26 expression in the subject. In some aspects, this detecting step allows for confirmation that in response to the alternating electric fields, an increase in aSMA, FAP, PDGFRO, ITDB1, CAV1, and/or CD26 expression is detected thus requiring the step of administering a CAF inhibitor.

In some aspects, administering a CAF inhibitor is performed 1, 2, 3, 4, 5, 6, 7, 8, 9, or 10 days after applying alternating electric fields. In some aspects, the amount of time to wait after applying alternating electric fields before administering a CAF inhibitor can depend on the amount of time it takes for the alternating electric fields to cause fibroblast activation or increase expression of CAF markers.

In some aspects, the target site comprises one or more cancer cells. In some aspects, the target site comprises one or more mesothelioma cells, ovarian cancer cells, cervical cancer cells, lung cancer cells, brain cancer cells, pancreatic cancer cells, breast cancer cells, hepatocellular cancer cells, renal cancer cells or colon cancer cells. In some aspects, the target site comprises cancer cells from any type of cancer.

In some aspects, a subject in need thereof is not simultaneously undergoing chemotherapy in combination with alternating electric fields. In some aspects, a subject in need thereof has not ever undergone chemotherapy prior to receiving the alternating electric fields. In some aspects, a subject in need thereof has not received chemotherapy within 1, 2, 3, 4, 5, 6, 7, 8, 9, 10, 11, or 12 months prior to receiving the alternating electric fields.

### D. Methods of Reducing/Preventing CAF Activation

Disclosed are methods of reducing or preventing CAF activation comprising applying alternating electric fields, to a population of cells comprising one or more fibroblasts; and contacting a CAF activation inhibitor to the population of cells.

In some aspects, reducing or preventing CAF activation can be achieved by inhibiting FAP, PDGFRb, Hsp90, Hedgehog, Hyaluronic acid formation, IL-1R, and/or LLRC15. Thus, in some aspects, a CAF activation inhibitor can be, but is not limited to, a fibroblast activation protein (FAP) inhibitor, a selective PDGFRb inhibitor, a selective Hedgehog inhibitor, a selective Hyaluronic acid depletion agent, an IL-1R inhibitor, a HSP90 inhibitor, or a LLRC15 inhibitor.

In some aspects, a FAP inhibitor can be, but is not limited to, small molecule inhibitors such as Talabostat or FAP-2286, Simlukafusp alfa (FAP-IL2v, R06874281/RG7461), SP-12786, Gemigliptin, FAPI-4, FAPI-2, OncoFAP, FAPI-34, TAM558, BR102910, or a neutralizing antibody against FAP such as TAM558. In some aspects, the FAP inhibitor can be one or more of those provided in which is incorporated herein by reference for its teaching of FAP inhibitors.

In some aspects, a selective PDGFRb inhibitor refers to an inhibitor that only binds to PDGFRb and does not bind to other PDGFR ligands. In some aspects, a selective PDGFRb inhibitor can be, but is not limited to, sPDGFRbIg, Crenolanib, Orantinib, Linifanib, CP-673451, SU16f, Tyrphostin AG 1296, Tyrphostin AG1433, SU4312, SU6668, AC710, JNJ 10198409, DMPQ, PD 166285, KG5, PDGFR tyrosine kinase inhibitor III, PDGFR-IN-1, Ansornitinib, PP58, GZD856 or Seralutinib.

In some aspects, a Hsp90 inhibitor can be, but is not limited to, XL888, Geldanamycin, Gedunin, Tanespimycin, Luminespib, Radicicol, 17-DMAG, EC144, Herbimycin A, Ganetespib, Onalespib, NVP-BEP800, SNX-2112, PF-04929113, KW-2478, NMS-E973, Zelavespib, Pseudolaric acid A, VER-49009, Pimitespib, CH538303, VER-50589, Cucurnitacin, HSP990, BIIV 021, CCT 018159,17-AAG. In some aspects, the Hsp90 inhibitor can be one or more of those provided in www.medchemexpress.com/search.html?q=hsp90&ft=&fa=&fp=&fsp=&ftag=&fsc=, which is incorporated herein by reference for its teaching of Hsp90 inhibitors.

In some aspects, a LLRC15 inhibitor can be, but is not limited to, ABBV-085.

In some aspects, the population of cells can be in vitro or in vivo. For example, applying alternating electric fields can include applying the alternating electric fields to a culture dish comprising a population of cells. In some aspects, applying alternating electric fields can include applying the alternating electric fields to a subject, wherein the population of cells is in the subject. In some aspects, applying alternating electric fields to a population of cells in a subject comprises applying alternating electric fields to a target site in a subject, wherein the target site comprises a population of cells. In some aspects, the target site comprises one or more cancer cells. Thus, in some aspects, the target site comprises one or more cancer cells. In some aspects, the target site comprises one or more mesothelioma cells, ovarian cancer cells, cervical cancer cells, lung cancer cells, brain cancer cells, pancreatic cancer cells, breast cancer cells, hepatocellular cancer cells, renal cancer cells or colon cancer cells. In some aspects, the target site comprises cancer cells from any type of cancer.

In some aspects, the alternating electric field is applied before, after, or simultaneously with the CAF activation inhibitor. In some aspects, the step of applying the alternating electric fields begins at least one hour before a CAF activation inhibitor is added. In some aspects, the step of applying the alternating electric fields begins at least 30 minutes before a CAF activation inhibitor is added. In some aspects, applying the alternating electric fields simultaneously can mean applying within 5, 10, 15, 20, 25, 30, 35, 40, 45, 50, 55, or 60 minutes before or after adding a CAF activation inhibitor. In some aspects, the alternating electric fields can be applied and the CAF activation inhibitor administered at least 1, 2, 3, 4, 5, 6, 7, 8, 9, 10, 11, 12, 13, 14, 15, 16, 17, 18, 19, 20, 21, 22, 23, or 24 hrs from each other.

In some aspects, the CAF activation inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the effects of the CAF activation inhibitor can be determined by counting the number of activated fibroblasts and/or detecting the presence or absence of CAF markers. For example, in some aspects, the CAF activation inhibitor decreases the number of activated fibroblasts. In some aspects, the CAF activation inhibitor inhibits or decreases FAP, alpha smooth muscle actin (aSMA), PDGFRb expression, Hedgehog expression, or hyaluronic acid formation.

In some aspects, the CAF activation inhibitor blocks upregulation of FAP, aSMA and/or PDGFRb expression caused by alternating electric fields. In some aspects, the CAF inhibitor blocks signaling through FAP, aSMA and/or PDGFRb caused by alternating electric fields.

In some aspects, the CAF activation inhibitor prevents PDGFRb from interacting with or binding to PDGF. For example, in some aspects, the CAF inhibitor binds to or interacts with PDGFRb thus preventing PDGFRb from interacting with or binding to its ligand, PDGF.

In some aspects, the CAF inhibitor prevents Hsp90 ATPase activity. In some aspects, Hsp90 ATPase activity upregulates fibroblast activation therefore preventing or inhibiting Hsp90 ATPase activity can inhibit CAF activation.

In some aspects, extracellular Hsp90a interacts with ER stress (which can be caused by alternating electric fields) to promote fibroblasts activation. Therefore, in some aspects, a CAF inhibitor that is an Hsp90 inhibitor can prevent or inhibit CAF activation.

In some aspects, the frequency of the alternating electric field is between 100 and 500 kHz. In some aspects, the frequency of the alternating electric fields is between 50 kHz and 1 MHz. In some aspects, the frequency of the alternating electric field is about 150 kHz, about 200 kHz, or about 250 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 150 and 300 kHz, between 180 and 220 kHz, or between 210 and 400 kHz. In some aspects, the frequency of the alternating electric fields can be 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the field strength can be about or at least 1.75 V/cm RMS. In some embodiments the field strength is about or at least 1 V/cm RMS. In some aspects, the alternating electric field has a field strength of about or at least 0.9 V/cm RMS. In some aspects, the alternating electric field has a field strength of any of those described herein. In other embodiments, combinations of field strengths are applied, for example combining two or more frequencies at the same time, and/or applying two or more frequencies at different times.

In some aspects, the alternating electric field can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric field can be applied every day for a two hour duration. For example, the alternating electric field can be applied for at least 4 hours per day, at least 8 hours per day, at least 12 hours per day, at least 16 hours per day, or at least 20 hours per day. In some aspects the alternating electric field can be applied for at least 4, 8, 12, 16, or 20 hours per day for at least 2 days. In some aspects the alternating electric field can be applied for at least 4, 8, 12, 16, or 20 hours per day for at least 3 days. In some aspects the alternating electric fields can be applied for at least 4, 8, 12, 16, or 20 hours per day for at least 7 days.

In some aspects, the consecutive exposure may last for at least 6 hours, at least 12 hours, at least 24 hours, at least 36 hours, at least 48 hours, or at least 72 hours or more.

In some aspects, the cumulative exposure may last for at least 42 hours, at least 84 hours, at least 168 hours, at least 250 hours, at least 400 hours, at least 500 hours, at least 750 hours, or more.

In some aspects, after applying an alternating electric field and prior to contacting the cells with a CAF activation inhibitor, the method can further comprise a step of detecting an increase in aSMA, FAP, PDGFRP, ITDB1, hedgehog, hyaluronic acid, Hsp90 and/or CD26, and/or CAV1 expression in the cells. In some aspects, this detecting step allows for confirmation that in response to the alternating electric fields, an increase in aSMA, FAP, PDGFRP, ITDB1, hedgehog, hyaluronic acid, Hsp90, CD26, and/or CAV1 expression is detected thus requiring the step of contacting with a CAF inhibitor.

In some aspects, administering a CAF activation inhibitor is performed 1, 2, 3, 4, 5, 6, 7, 8, 9, or 10 days after applying alternating electric fields. In some aspects, the amount of time to wait after applying alternating electric fields before contacting with a CAF activation inhibitor can depend on the amount of time it takes for the alternating electric fields to cause fibroblast activation or increase expression of CAF markers.

In some aspects, if the population of cells is in a subject, the subject is not simultaneously undergoing chemotherapy in combination with alternating electric fields. In some aspects, if the population of cells is in a subject, the subject has not ever undergone chemotherapy prior to receiving the alternating electric fields. In some aspects, if the population of cells is in a subject, the subject has not received chemotherapy within 1, 2, 3, 4, 5, 6, 7, 8, 9, 10, 11, or 12 months prior to receiving the alternating electric fields.

### E. Methods of Reducing CAF Signaling

Disclosed are methods of reducing CAF signaling comprising applying an alternating electric field, to a population of cells comprising one or more fibroblasts; and contacting a cancer associated fibroblast (CAF) signaling inhibitor to the population of cells.

In some aspects, a CAF signaling inhibitor can be, but is not limited to, a Lox (lysyl oxidase) inhibitor or LoxL (lysyl oxidase like) inhibitor. In some aspects, LoxL inhibitors can be, but are not limited to, monoclonal antibodies against Lysyl oxidase like-1/2/3/4 such as simtuzumab, PXS-5120A, PAT-1251, PXS-4787, PXS-6302, Anti-LOXL 1-4 antibody [LA3] or a small molecule inhibitor such as PXS-5153A. In some aspects, a LOX inhibitor can be, but is not limited to, LOX-IN-3, β-Aminopropionitrile (BAPN), CCT365623, PXS-6302, PXS-5505, mIR142-3p[LA4] or PXS-4787. In some aspects, the LOX inhibitor can be one or more of the inhibitors provided in www.medchemexpress.com/search.html?q=Lysyl+oxidase&ft=&fa=&fp=&fsp=&ftag=&fsc=& type=inhibitors-and-agonists, which is incorporated herein by reference for its teaching of LOX inhibitors.

In some aspects, a CAF signaling inhibitor can be, but is not limited to, a CXCR4 inhibitor or a CXCL-12 inhibitor. CXCL12-CXCR4 axis antagonism has been tested in clinical trials since CXCL12 is secreted by CAFs and promotes tumorigenesis. CXCR4 inhibitors include, for example, Olaptesed (NOX-A12), BL-8040 CXCR4 Antagonist, TC14012, KRH 2955, ITIt, Plerixafor (AMD 3100), WZ811, USL311, LY2510924, MSX-127, MSX-130, AMD 3465, CTCE 9908, Tannic acid, Motixafortide (BL-8040), MSX-122, Balixafortide (POL6326), ATI 2341 or CXCR4 antibody. CXCL12 inhibitors include, for example, UNBS5162, LIT-927, or CXCL12 antibody.

For example, applying an alternating electric field can include applying the alternating electric field to a culture dish comprising a population of cells. In some aspects, applying an alternating electric field can include applying the alternating electric field to a subject, wherein the population of cells is in the subject. In some aspects, applying an alternating electric field to a population of cells in a subject comprises applying alternating electric fields to a target site in a subject, wherein the target site comprises a population of cells. In some aspects, the target site comprises one or more cancer cells. Thus, in some aspects, the population of cells is a population of cancer cells. In some aspects, the target site comprises one or more cancer cells. In some aspects, the target site comprises one or more mesothelioma cells, ovarian cancer cells, cervical cancer cells, lung cancer cells, brain cancer cells, pancreatic cancer cells, breast cancer cells, hepatocellular cancer cells, renal cancer cells or colon cancer cells. In some aspects, the target site comprises cancer cells from any type of cancer.

In some aspects, the alternating electric field is applied before, after, or simultaneously with the CAF signaling inhibitor. In some aspects, the step of applying the alternating electric fields begins at least one hour before a CAF signaling inhibitor is added. In some aspects, the step of applying the alternating electric fields begins at least 30 minutes before a CAF signaling inhibitor is added. In some aspects, applying the alternating electric field simultaneously can mean applying within 5, 10, 15, 20, 25, 30, 35, 40, 45, 50, 55, or 60 minutes before or after adding a CAF signaling inhibitor. In some aspects, the alternating electric fields can be applied and the CAF signaling inhibitor administered at least 1, 2, 3, 4, 5, 6, 7, 8, 9, 10, 11, 12, 13, 14, 15, 16, 17, 18, 19, 20, 21, 22, 23, or 24 hrs from each other.

In some aspects, the CAF signaling inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the effects of the CAF signaling inhibitor can be determined by detecting downstream signaling events. In some aspects, the CAF signaling inhibitor can block ligands or receptors involved in CAF signaling thereby preventing a signaling event.

In some aspects, the CAF signaling inhibitor prevents Lox or Loxl from covalently cross-linking collagen and elastin in extracellular matrix (ECM). The lysyl oxidase (LOX) family of enzymes plays a critical role in the formation, maturation, and remodeling of extracellular matrix (ECM) which supports tumor growth and metastasis. Therefore, in some aspects, inhibiting signaling via Lox or Loxl can treat cancers.

In some aspects, CXCL12-CXCR4 axis antagonism has been tested in clinical trials since CXCL12 is secreted by CAFs and promotes tumorigenesis. CXCR4 Inhibitors are for example, Olaptesed (NOX-A12), BL-8040 CXCR4 Antagonist, TC14012, KRH 2955, ITlt, Plerixafor (AMD 3100), WZ811, USL311, LY2510924, MSX-127, MSX-130, AMD 3465, CTCE 9908, Tannic acid, Motixafortide (BL-8040), MSX-122, Balixafortide (POL6326), ATI 2341or CXCR4 antibody. CXC112 Inhibitors are for example UNBS5162, LIT-927, or CXCL12 antibody.

In some aspects, the frequency of the alternating electric field is between 100 and 500 kHz. In some aspects, the frequency of the alternating electric fields is between 50 kHz and 1 MHz. In some aspects, the frequency of the alternating electric field is about 150 kHz, about 200 kHz, or about 250 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 150 and 300 kHz, between 180 and 220 kHz, or between 210 and 400 kHz. In some aspects, the frequency of the alternating electric fields can be 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the field strength can be about or at least 1.75 V/cm RMS. In some embodiments the field strength is about or at least 1 V/cm RMS. In some aspects, the alternating electric field has a field strength of about or at least 0.9 V/cm RMS. In some aspects, the alternating electric field has a field strength of any of those described herein. In other embodiments, combinations of field strengths are applied, for example combining two or more frequencies at the same time, and/or applying two or more frequencies at different times.

In some aspects, the alternating electric field can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric field can be applied every day for a two hour duration. For example, the alternating electric field can be applied for at least 4 hours per day, at least 8 hours per day, at least 12 hours per day, at least 16 hours per day, or at least 20 hours per day. In some aspects the alternating electric field can be applied for at least 4, 8, 12, 16, or 20 hours per day for at least 2 days. In some aspects the alternating electric field can be applied for at least 4, 8, 12, 16, or 20 hours per day for at least 3 days. In some aspects the alternating electric fields can be applied for at least 4, 8, 12, 16, or 20 hours per day for at least 7 days.

In some aspects, the consecutive exposure may last for at least 6 hours, at least 12 hours, at least 24 hours, at least 36 hours, at least 48 hours, or at least 72 hours or more.

In some aspects, the cumulative exposure may last for at least 42 hours, at least 84 hours, at least 168 hours, at least 250 hours, at least 400 hours, at least 500 hours, at least 750 hours, or more.

In some aspects, after applying an alternating electric field and prior to contacting with a CAF inhibitor, the method can further comprise a step of detecting an increase in aSMA, FAP, PDGFRP, hedgehog, hyaluronic acid, Hsp90, CD26, and/or CAV1 expression in the cells. In some aspects, this detecting step allows for confirmation that in response to the alternating electric field, an increase in aSMA, FAP, PDGFRP, hedgehog, hyaluronic acid, Hsp90, CD26, and/or CAV1 expression is detected thus requiring the step of administering a CAF inhibitor.

In some aspects, administering a CAF signaling inhibitor is performed 1, 2, 3, 4, 5, 6, 7, 8, 9, or 10 days after applying an alternating electric field. In some aspects, the amount of time to wait after applying an alternating electric field before administering a CAF signaling inhibitor can depend on the amount of time it takes for the alternating electric field to cause fibroblast activation or increase expression of CAF markers.

In some aspects, the target site comprises one or more cancer cells. In some aspects, the target site comprises one or more mesothelioma cells, ovarian cancer cells, cervical cancer cells, lung cancer cells, brain cancer cells, pancreatic cancer cells, breast cancer cells, hepatocellular cancer cells, renal cancer cells or colon cancer cells. In some aspects, the target site comprises cancer cells from any type of cancer.

In some aspects, if the population of cells is in a subject, the subject is not simultaneously undergoing chemotherapy in combination with alternating electric fields. In some aspects, if the population of cells is in a subject, the subject has not ever undergone chemotherapy prior to receiving the alternating electric fields. In some aspects, if the population of cells is in a subject, the subject has not received chemotherapy within 1, 2, 3, 4, 5, 6, 7, 8, 9, 10, 11, or 12 months prior to receiving the alternating electric fields.

### F. Methods of Altering Tumors

Disclosed are methods of decreasing tumor growth in a subject in need thereof comprising applying alternating electric fields, to a target site of the subject in need thereof; and administering a CAF inhibitor to the subject in need thereof.

Disclosed are methods of decreasing tumor invasion in a subject in need thereof comprising applying alternating electric fields, to a target site of the subject in need thereof; and administering a CAF activation inhibitor to the subject in need thereof.

Disclosed are methods of decreasing tumor angiogenesis in a subject in need thereof comprising applying alternating electric fields, to a target site of the subject in need thereof; and administering a CAF inhibitor to the subject in need thereof.

Disclosed are methods of decreasing tumor metastasis in a subject in need thereof comprising applying alternating electric fields, to a target site of the subject in need thereof; and administering a CAF activation inhibitor such as FAP inhibitor, a selective platelet derived growth factor receptor beta (PDGFRb) inhibitor, Hedgehog inhibitor, Hyaluronic acid depletion, Hsp90 inhibitor, IL-1R inhibitor, or an LLRC15 inhibitor to the subject in need thereof.

In some aspects, the target site comprises one or more cancer cells. In some aspects, the target site comprises one or more mesothelioma cells, ovarian cancer cells, cervical cancer cells, lung cancer cells, brain cancer cells, pancreatic cancer cells, breast cancer cells, hepatocellular cancer cells, renal cancer cells or colon cancer cells. In some aspects, the target site comprises cancer cells from any type of cancer.

In some aspects, a CAF inhibitor can be a CAF activation inhibitor or a CAF signaling inhibitor. Thus, in some aspects, a CAF inhibitor can prevent or reduce CAF activation or can prevent or reduce signaling from CAFs.

In some aspects, a CAF activation inhibitor can be, but is not limited to, a fibroblast activation protein (FAP) inhibitor, a selective platelet derived growth factor receptor beta (PDGFRb) inhibitor, a HSP90 inhibitor, Hyaluronic acid depletion, Hedgehog inhibitor, IL-1R inhibitor or an LLRC15 inhibitor.

In some aspects, a FAP inhibitor can be, but is not limited to, small molecule inhibitors such as Talabostat or FAP-2286, Simlukafusp alfa (FAP-IL2v, R06874281/RG7461), SP-12786, Gemigliptin, FAPI-4, FAPI-2, OncoFAP, FAPI-34, TAM558, BR102910, or a neutralizing antibody against FAP such as TAM558. In some aspects, the FAP inhibitor can be one or more of those provided in which is incorporated herein by reference for its teaching of FAP inhibitors.

In some aspects, a selective PDGFRb inhibitor refers to an inhibitor that only binds to PDGFRb and does not bind to other PDGFR ligands. In some aspects, a selective PDGFRb inhibitor can be, but is not limited to, sPDGFRbIg, Crenolanib, Orantinib, Linifanib, CP-673451, SU16f, Tyrphostin AG 1296, Tyrphostin AG1433, SU4312, SU6668, AC710, JNJ 10198409, DMPQ, PD 166285, KG5, PDGFR tyrosine kinase inhibitor III, PDGFR-IN-1, Ansornitinib, PP58, GZD856 or Seralutinib.

In some aspects, a Hsp90 inhibitor can be, but is not limited to, XL888, Geldanamycin, Gedunin, Tanespimycin, Luminespib, Radicicol, 17-DMAG, EC144, Herbimycin A, Ganetespib, Onalespib, NVP-BEP800, SNX-2112, PF-04929113, KW-2478, NMS-E973, Zelavespib, Pseudolaric acid A, VER-49009, Pimitespib, CH538303, VER-50589, Cucurnitacin, HSP990, BIIV 021, CCT 018159,17-AAG. In some aspects, the Hsp90 inhibitor can be one or more of the inhibitors provided in www.medchemexpress.com/search.html?q=hsp90&ft=&fa=&fp=&fsp=&ftag=&fsc=, which is incorporated herein by reference for its teaching of Hsp90 inhibitors.

In some aspects, a LLRC15 inhibitor can be, but is not limited to, ABBV-085.

In some aspects, a Hedgehog inhibitor can be, but is not limited to IPI-926, Vismodegib, KAAD-Cyclopamine, Cyclopamine, Robotnikinin, 3-epi-Vitamin D3, Sonidegib (NVP-LDE225), Jervine, HPI 1, Dynarrestin, TAK-441, TPB15, JK184, Ciliobrevin A, Hh-Ag1.5, SANT 2, MK-4101, MRT 10, U 18666A, CUR61414 or Itraconazole (R51211). In some aspects, the Hedgehog inhibitor can be one or more of the inhibitors provided in www.medchemexpress.com/search.html?q=hedgehog&ft=&fa=&fp=&fsp=&ftag=&fsc, which is incorporated herein by reference for its teaching of Hedgehog inhibitors.

In some aspects, a hyaluronic acid depletion agent can be, but is not limited to PEGPH20, 4-MU, Hyaluronidase or an angiotensin receptor agent that attenuates Hyaluronan secretion such as Losartan, Mopivabil, Pratosartan, Mepixetil, LY285434, Azilsartan mopivanil, Tasosartan, ZD 7155, BIBS 39 or CGP 48369 [LA3]. In some aspects, the hyaluronic acid depletion agent can be one or more of the inhibitors provided in www.tocris.com/search?keywords=angiotensin+inhibitor or www.medchemexpress.com/search.html?q=angiotensin+II+receptor+antagonist&ft=&fa=&fp=&fsp=&ftag=&fsc=, which is incorporated herein by reference for its teaching of hyaluronic acid depletion.

In some aspects, an IL-1R inhibitor can be, but is not limited to Anakinra, AF12198 or Diacerein.

In some aspects, a CAF signaling inhibitor can be, but is not limited to, a Lox (lysyl oxidase) inhibitor or LoxL (lysyl oxidase like[LA4]) inhibitor. In some aspects, LoxL inhibitors can be, but are not limited to, monoclonal antibodies against Lysyl oxidase like-2/3 such as simtuzumab, PXS-5120A, PAT-1251, PXS-4787, PXS-6302, Anti-LOXL 1-4 antibody [LA5] or a small molecule inhibitor such as PXS-5153A. In some aspects, a LOX inhibitor can be, but is not limited to, LOX-IN-3, β-Aminopropionitrile (BAPN), CCT365623, PXS-6302, PXS-5505, mIR142-3p[LA6] or PXS-4787. In some aspects, the LOX inhibitor can be one or more of those provided in www.medchemexpress.com/search.html?q=Lysyl+oxidase&ft=&fa=&fp=&fsp=&ftag=&fsc=& type=inhibitors-and-agonists, which is incorporated herein by reference for its teaching of LOX inhibitors [LA7].

In some aspects, a CAF signaling inhibitor can be, but is not limited to, a CXCR4 inhibitor or a CXCL12 inhibitor. CXCL12-CXCR4 axis antagonism has been tested in clinical trials since CXCL12 is secreted by CAFs and promotes tumorigenesis. CXCR4 inhibitors include, for example, Olaptesed (NOX-A12), BL-8040 CXCR4 Antagonist, TC14012, KRH 2955, IT1t, Plerixafor (AMD 3100), WZ811, USL311, LY2510924, MSX-127, MSX-130, AMD 3465, CTCE 9908, Tannic acid, Motixafortide (BL-8040), MSX-122, Balixafortide (POL6326), ATI 2341or CXCR4 antibody. CXCL12 inhibitors include, for example, UNBS5162, LIT-927, or CXCL12 antibody.

In some aspects, the alternating electric field is applied before, after, or simultaneously with administering the CAF inhibitor. In some aspects, the step of applying the alternating electric fields begins at least one hour before a CAF activation inhibitor or CAF signaling inhibitor. In some aspects, the step of applying the alternating electric fields begins at least 30 minutes before a CAF activation inhibitor or CAF signaling inhibitor. In some aspects, applying the alternating electric fields simultaneously can mean applying within 5, 10, 15, 20, 25, 30, 35, 40, 45, 50, 55, or 60 minutes before or after administering a CAF activation inhibitor or CAF signaling inhibitor. In some aspects, the alternating electric fields can be applied and the CAF activation inhibitor or CAF signaling inhibitor administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the CAF inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the effects of the CAF activation inhibitor can be determined by counting the number of activated fibroblasts and/or detecting the presence or absence of CAF markers. For example, in some aspects, in some aspects, the CAF activation inhibitor decreases the number of activated fibroblasts. In some aspects, the CAF activation inhibitor inhibits or decreases FAP, alpha smooth muscle actin (aSMA), hedgehog, hyaluronic acid, or PDGFRb expression.

In some aspects, the effects of the CAF signaling inhibitors can be determined by detecting downstream signaling events. In some aspects, the CAF signaling inhibitors can block ligands or receptors involved in CAF signaling thereby preventing a signaling event.

In some aspects, the CAF inhibitor blocks upregulation of FAP, aSMA, hedgehog, hyaluronic acid, and/or PDGFRb expression caused by alternating electric fields. In some aspects, the CAF inhibitor blocks signaling through FAP, aSMA, hedgehog, hyaluronic acid, and/or PDGFRb caused by alternating electric fields.

In some aspects, the CAF inhibitor prevents PDGFRb from interacting with or binding to PDGF. For example, in some aspects, the CAF inhibitor binds to or interacts with PDGFRb thus preventing PDGFRb from interacting with or binding to its ligand, PDGF.

In some aspects, the CAF inhibitor prevents Hsp90 ATPase activity. In some aspects, Hsp90 ATPase activity upregulates fibroblast activation therefore preventing or inhibiting Hsp90 ATPase activity can inhibit CAF activation.

In some aspects, extracellular Hsp90a interacts with ER stress (which can be caused by alternating electric fields) to promote fibroblasts activation. Therefore, in some aspects, a CAF inhibitor that is an Hsp90 inhibitor can prevent or inhibit CAF activation.

In some aspects, the CAF inhibitor prevents Lox or Loxl from covalently cross-linking collagen and elastin in extracellular matrix (ECM). The lysyl oxidase (LOX) family of enzymes plays a critical role in the formation, maturation, and remodeling of extracellular matrix (ECM) which supports tumor growth and metastasis. Therefore, in some aspects, inhibiting signaling via Lox or Loxl can treat cancers.

In some aspects, Hedgehog pathway intersects with CAF heterogeneity to promote fibroblasts activation. Therefore, in some aspects, a Hedgehog inhibitor can prevent or inhibit CAF activation.

In some aspects, inhibition of hyaluronic acid formation inhibits binding of hyaluronic acid to tumor cells thereby inhibiting the encouragement of CAFs to migrate and invade the tumor and promote tumorigenesis.

In some aspects, inhibition of angiotensin inhibits hyaluronic acid formation which inhibits binding of hyaluronic acid to tumor cells thereby encouraging CAFs to migrate and invade the tumor and promote tumorigenesis.

In some aspects, inhibition of IL-1R inhibits binding to IL-1R and inhibits signal transduction that leads to activation of CAFs.

In some aspects, the frequency of the alternating electric field is between 100 and 500 kHz. In some aspects, the frequency of the alternating electric fields is between 50 kHz and 1 MHz. In some aspects, the frequency of the alternating electric field is about 150 kHz, about 200 kHz, or about 250 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 150 and 300 kHz, between 180 and 220 kHz, or between 210 and 400 kHz. In some aspects, the frequency of the alternating electric fields can be 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the field strength can be about or at least 1.75 V/cm RMS. In some embodiments the field strength is about or at least 1 V/cm RMS. In some aspects, the alternating electric field has a field strength of about or at least 0.9 V/cm RMS. In some aspects, the alternating electric field has a field strength of any of those described herein. In other embodiments, combinations of field strengths are applied, for example combining two or more frequencies at the same time, and/or applying two or more frequencies at different times.

In some aspects, the alternating electric field can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric field can be applied every day for a two hour duration. For example, the alternating electric field can be applied for at least 4 hours per day, at least 8 hours per day, at least 12 hours per day, at least 16 hours per day, or at least 20 hours per day. In some aspects the alternating electric field can be applied for at least 4, 8, 12, 16, or 20 hours per day for at least 2 days. In some aspects the alternating electric field can be applied for at least 4, 8, 12, 16, or 20 hours per day for at least 3 days. In some aspects the alternating electric fields can be applied for at least 4, 8, 12, 16, or 20 hours per day for at least 7 days.

In some aspects, the consecutive exposure may last for at least 6 hours, at least 12 hours, at least 24 hours, at least 36 hours, at least 48 hours, or at least 72 hours or more.

In some aspects, the cumulative exposure may last for at least 42 hours, at least 84 hours, at least 168 hours, at least 250 hours, at least 400 hours, at least 500 hours, at least 750 hours, or more.

In some aspects, the disclosed methods of decreasing tumor growth, decreasing tumor invasion, decreasing tumor angiogenesis, and/or decreasing tumor metastasis can further comprise administering a cancer therapeutic. In some aspects, the cancer therapeutic is a known cancer therapeutic other than the CAF inhibitor. For example, the cancer therapeutic can be, but is not limited to, chemotherapy, radiation, immunotherapy, or hormone therapy. In some aspects, the cancer therapeutic can be, but is not limited to, tyrosine kinase inhibitors, PI3K inhibitors, Akt inhibitors, anti VEGF inhibitors, FGF inhibitors.

In some aspects, the alternating electric fields are applied before, after, or simultaneously with administering the cancer therapeutic. In some aspects, the CAF inhibitor is applied before, after, or simultaneously with administering the cancer therapeutic. In some aspects, the CAF inhibitor and cancer therapeutic are administered simultaneously and the alternating electric fields are applied before or after the CAF activation inhibitor or CAF signaling inhibitor and cancer therapeutic.

In some aspects, after applying alternating electric fields and prior to administering a CAF inhibitor, the method can further comprise a step of detecting an increase in aSMA, FAP, PDGFRP, and/or CD26 expression in the subject. In some aspects, this detecting step allows for confirmation that in response to the alternating electric fields, an increase in aSMA, FAP, PDGFRP, hyaluronic acid, Hsp90, hedgehog, LLRC15 and/or CD26 expression is detected thus requiring the step of administering a CAF inhibitor.

In some aspects, administering a CAF inhibitor is performed 1, 2, 3, 4, 5, 6, 7, 8, 9, or 10 days after applying alternating electric fields. In some aspects, the amount of time to wait after applying alternating electric fields before administering a CAF inhibitor can depend on the amount of time it takes for the alternating electric fields to cause fibroblast activation or increase expression of CAF markers.

### G. Compositions

Disclosed are compositions and formulations comprising one or more CAF activation inhibitors or CAF signaling inhibitors, or a combination thereof. In some embodiments the formulation further includes a pharmaceutically acceptable carrier or diluent. For example, disclosed are pharmaceutical compositions, comprising a CAF activation inhibitor or CAF signaling inhibitor and a pharmaceutically acceptable carrier. For example, disclosed are pharmaceutical compositions, comprising a FAP inhibitor, a selective PDGFRb inhibitor, a selective Hedgehog inhibitor, a selective Hyaluronic acid secretion inhibitor, IL-1R inhibitor, CXCR4 inhibitor, a HSP90 inhibitor, or a LLRC15 inhibitor, and a pharmaceutically acceptable carrier. Disclosed also are pharmaceutical compositions, comprising a CAF activation inhibitor or CAF signaling inhibitor and a pharmaceutically acceptable diluent.

In some aspects, the CAF activation inhibitor or CAF signaling inhibitor can be administered with a pharmaceutically acceptable carrier and/or diluent in any of the disclosed methods.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidylcholine (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, P A 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), gels (including hydrogels), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

### 1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, tri-alkyl and aryl amines and substituted ethanolamines.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of CAF inhibitors and one or more materials for delivering alternating electric fields, such as the Optune system. For example disclosed are kits comprising one or more of a CAF activation inhibitor or CAF signaling inhibitor and one or more materials for delivering alternating electric fields, such as the Optune system. In some aspects, the kits can also include a cancer therapeutic.

### Examples

Current data shows that TTFields increase activation of fibroblasts in vivo and in vitro.

CAFs are fibroblast that resides within the tumor tissue expressing and secreting proteins and by doing so they influence remodeling of the tumor. Activation of CAFs can promote tumor growth. CAFs usually express FAP (fibroblast activation protein), Alpha smooth muscle actin (aSMA), Platelet derived growth factor receptor beta (PDGFRb), integrin beta 1(ITDB1), CD26 and caveolin-1 (CAV1).

Through multiple pathways, activated CAFs can promote tumor growth, angiogenesis, invasion and metastasis. CAF activation can support tumor growth by secretion of various cytokines and interaction with tumor cells and immune cells

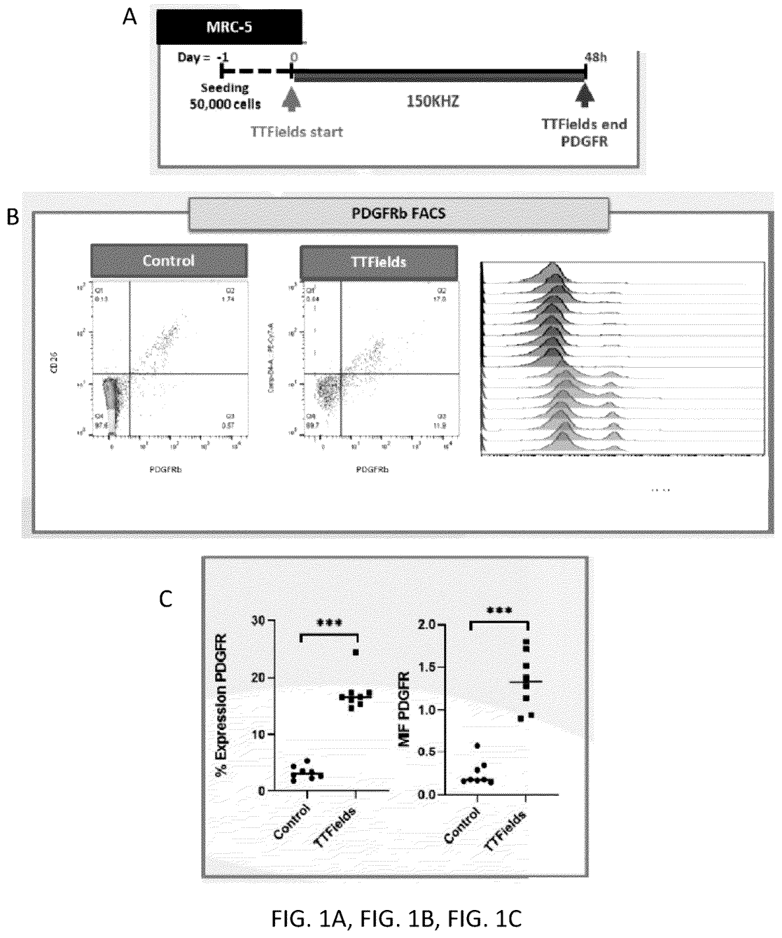

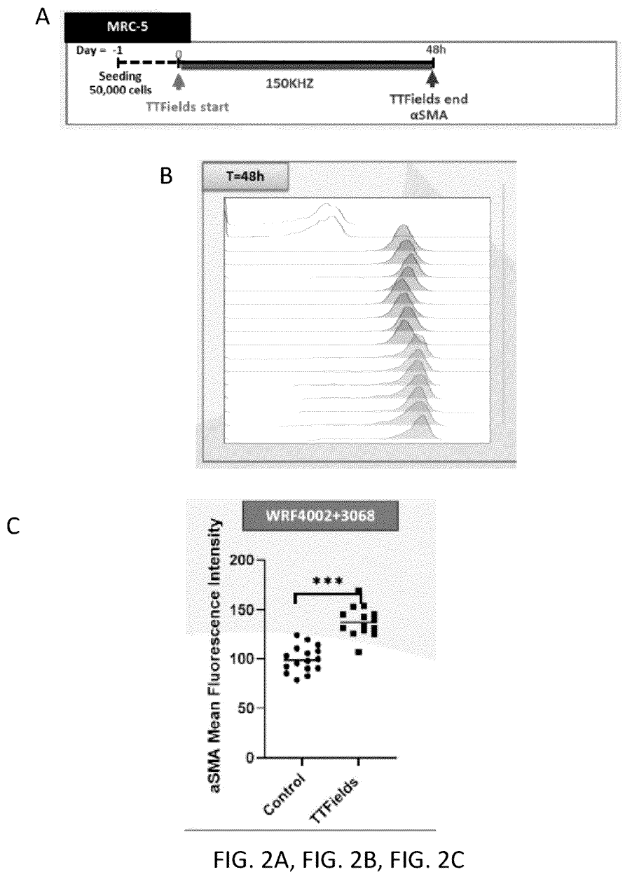

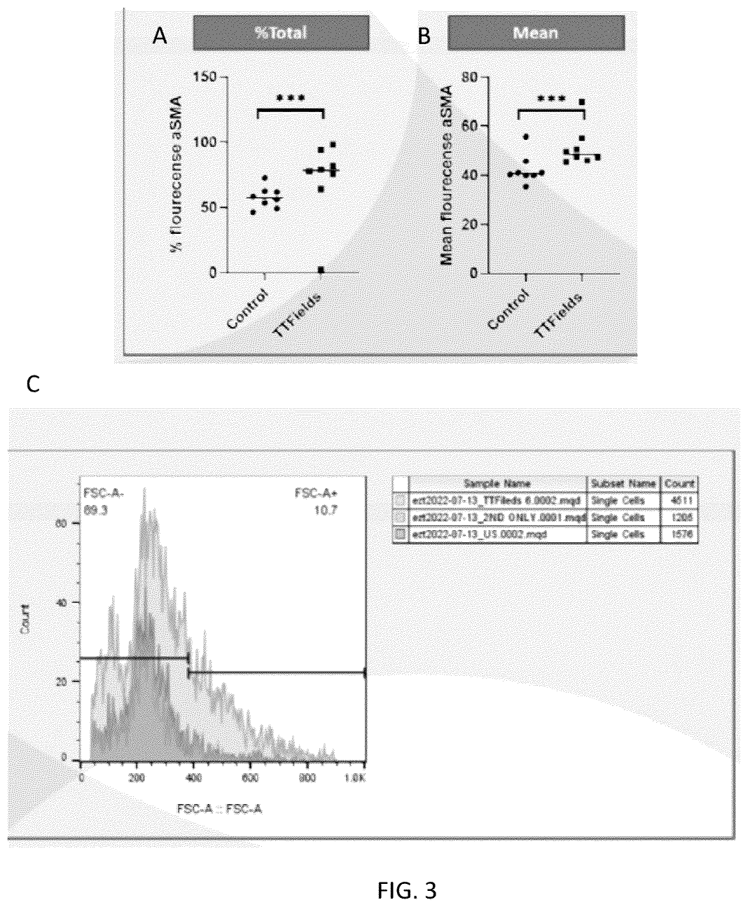

Application of TTFields for 48 hours in MRC5 lung fibroblasts resulted in up regulation of PDGFRb (FIG. 1A—schematic of experiment; FIG. 1B—FACS analysis; FIG. 1C Quantification of PDGFRb+percentage of cells and PDGRFb mean fluorescence intensity (MIF)) and aSMA (FIG. 2A—schematic of experiment; FIG. 2B—Application of TTFields for 48 hours in MRC5 lung fibroblasts resulted in up regulation of alpha SMA (aSMA) as seen by FACS analysis and MFI graph (results presented are from two individual experiments); FIG. 2C Quantification of aSMA mean fluorescence intensity expression in lung fibroblasts (MRC-5), indicating activation of fibroblast following 48 hours and of treatment. FIG. 3 Application of TTFields for 48 hours in MRC5 lung fibroblasts resulted in FIG. 3A an increase in percentage of fibroblasts expression aSMA and FIG. 3B—up regulation of alpha SMA (aSMA) as seen by FIG. 3C FACS analysis and MFI graph

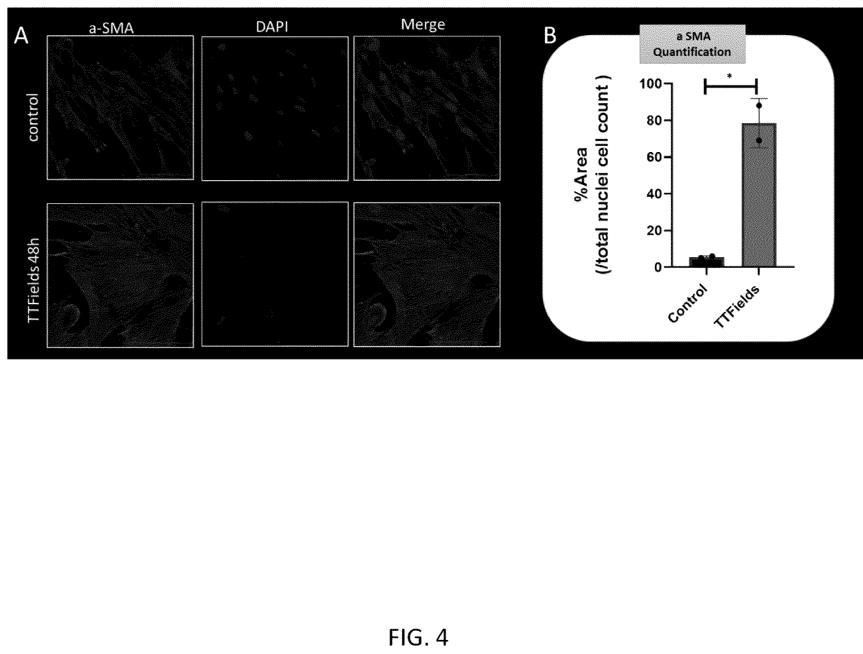

Thus, FIG. 3 shows in detail the results presented in FIG. 2C where not only the amount of cells expressing aSMA is increased but also the mean fluorescent intensity (MFI) is increased per cell indicating that cells express more aSMA. Confocal images of fibroblasts showing an increase in aSMA expression following 48 hours TTFields application as shown in FIG. 4A and quantified in FIG. 4B.

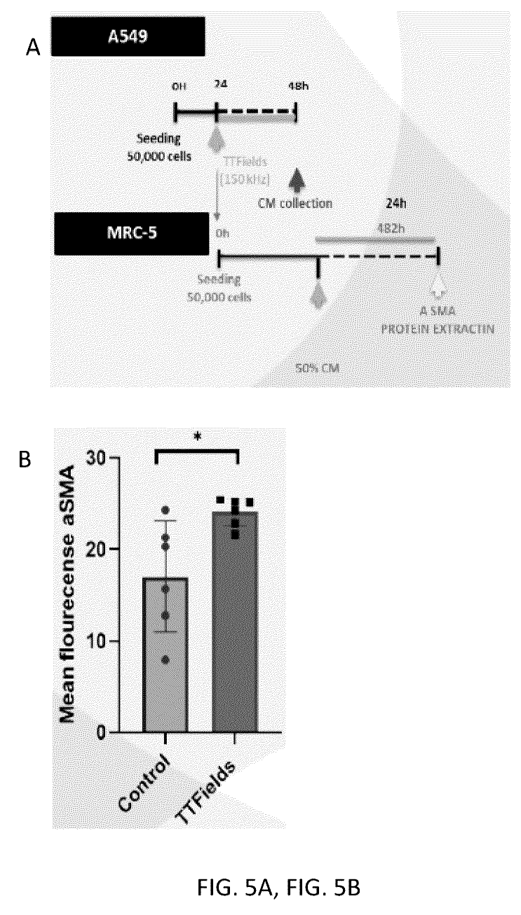

aSMA expression was increased in fibroblasts following incubation with conditioned medium (CM) derived from TTFields treated cancer cells (A549 cells—lung adenocarcinoma cells. FIG. 5A shows a schematic diagram of the experiments. FIG. 5B shows an increase in aSMA mean fluorescence intensity in TTFields treated fibroblasts compared to control.

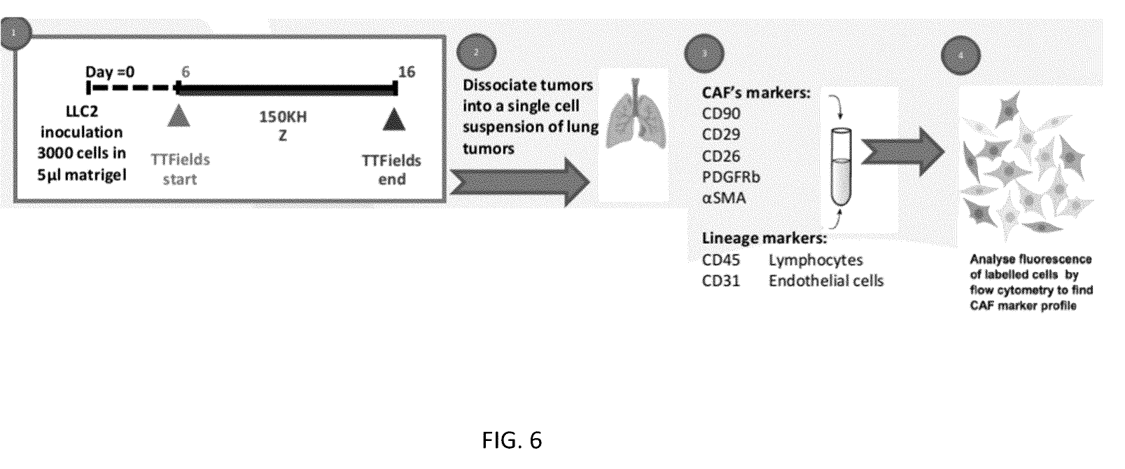

FIG. 6 shows the process of isolation and characterization of fibroblasts from lungs of LLC2 bearing mice following 10 days of TTFields application.

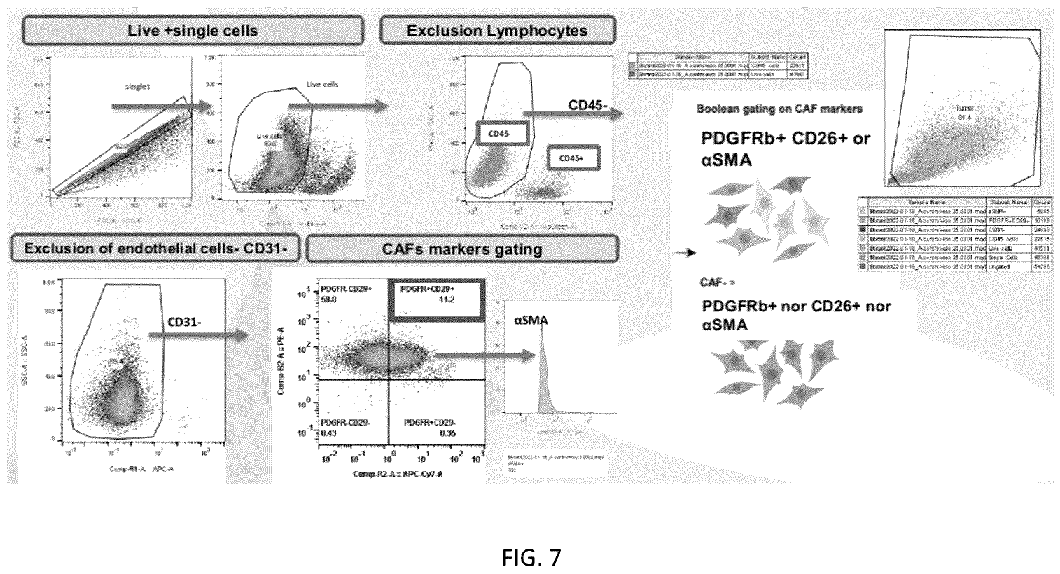

FIG. 7 shows a scheme of FACS analysis gating strategy to distinguish activated fibroblasts in samples of cells derived from tumor dissociation.

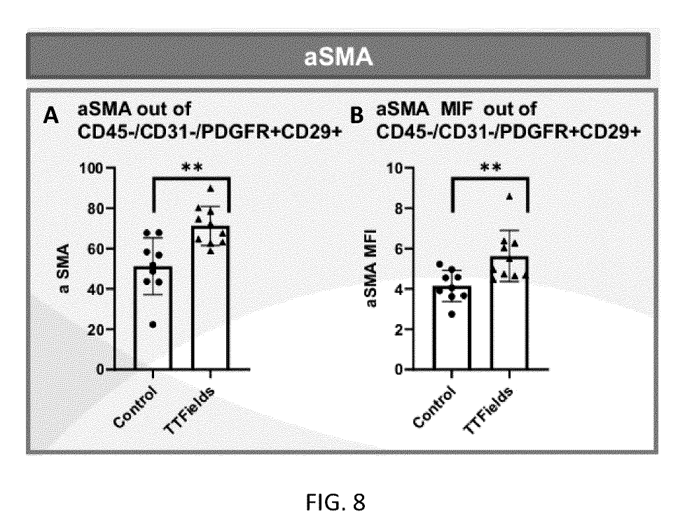

FIG. 8B shows aSMA expression is increased in CD45-/CD31-/PDGFR+CD29+ cells derived from tumors following TTFields application. FIG. 8A shows that statistically significant more CD45-/CD31-/PDGFR+CD29+ cells derived from tumors following TTFields express aSMA compared to control.

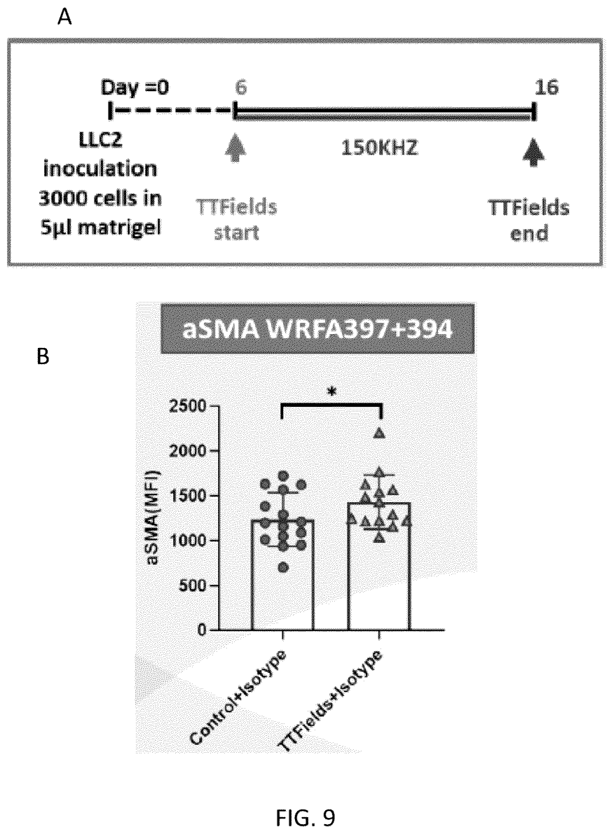

FIG. 9 shows aSMA expression is increased in CD45-/CD31-/PDGFR+CD29+ cells derived from tumors following TTFields application in two separate experiments.

The disclosed data shows that inhibiting CAFs signaling can be done by inhibiting PDGFRb. Targeting specific types of CAFs can be used for inhibition. Inhibiting FAP, Hsp90 Hedgehog, Hyaluronic acid secretion, IL-1R, CXCR4- CXCL12 and/or Lox and Loxl can all be used as CAF inhibition strategies.

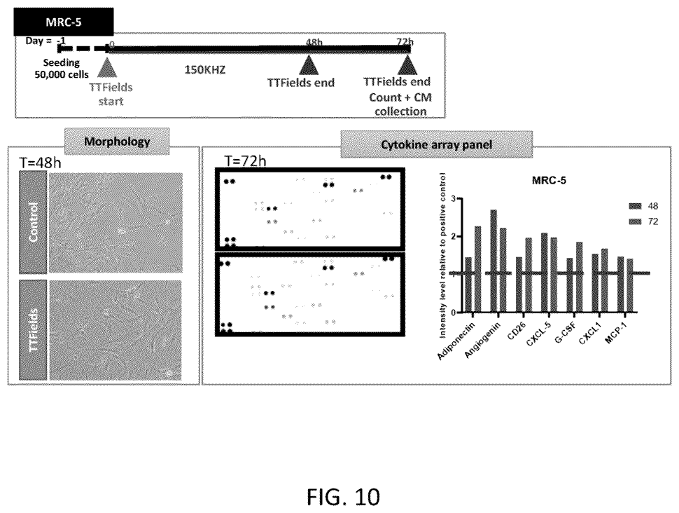

Also, secretory cytokine array assay of conditioned media derived from MRC5 following TTFields application for 72 hours at 150 kHz revealed secretion of MCP1 and CD26 which is a CAF marker indicating differentiation of fibroblast to CAFs (See FIG. 10).

In vivo, fibroblasts isolated from lungs of LLC2 bearing mice following TTFields treatment, exhibit CAF's phenotype: highly expressed aSMA.

Therefore, treatment with TTFields while inhibiting the activation of cancer associated fibroblast can result in better patient outcome.

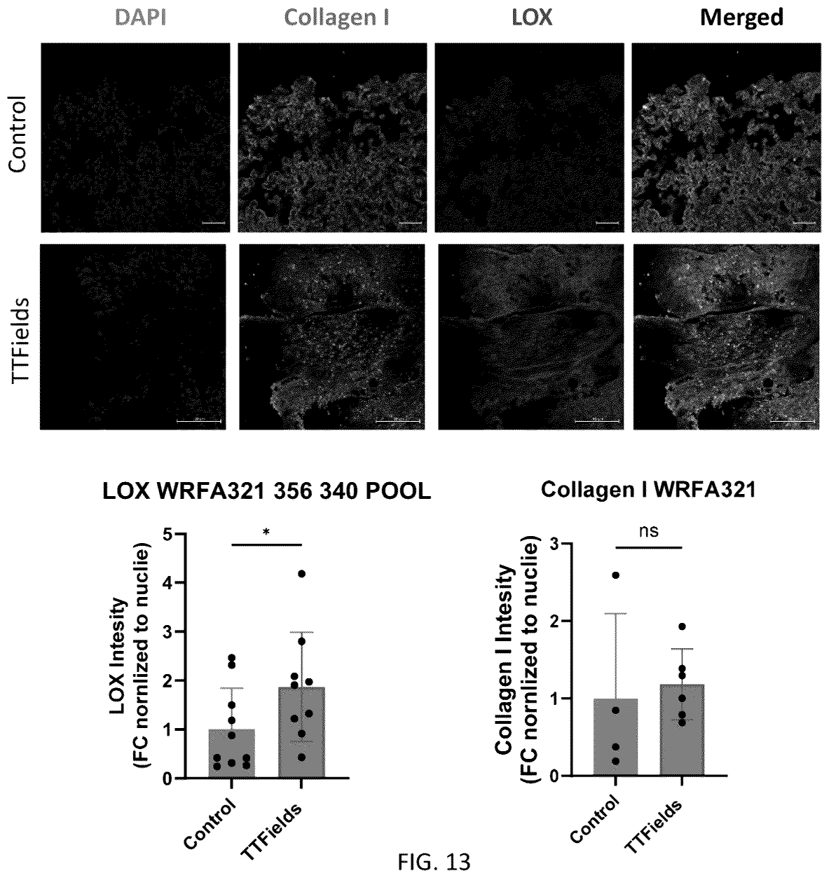

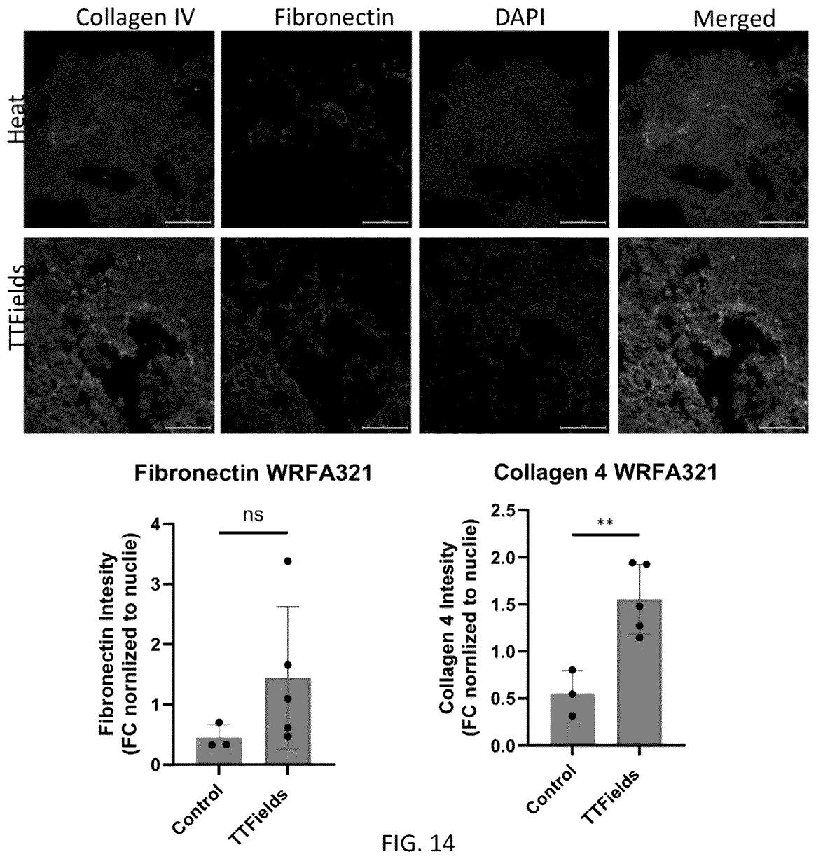

ECM remodeling following TTFields application was examined in orthotopic models of ovarian cancer (MOSE-L-FFL). TTFields were continuously applied for 10 days in a frequency of 200 kHz to ovarian tumor bearing mice. Treatment with TTFields significantly elevated LOX protein expression and Collagen 4 protein expression as visualized and quantified in tumor sections, with no significant change in Collagen 1 and Fibronectin protein expression within tumor sections (FIGS. 13 and 14).

TTFields application to mice: TTFields were applied through 2 pairs of arrays placed on the torso of the mice (ovarian mouse model).

Ex vivo Immunohistochemical analysis: Tumors were collected from Female C57B1/6 mice inoculated with MOSE-L-FFL ovarian carcinoma cells into the ovarian bursa, and treated with sham heat or 200 kHz TTFields for 10 consecutive days; Tumor sections were fixed in 4% PFA and embedded in OCT for ovary and examined immunohistochemically with an anti-LOX (1:100, NB100-2530), collagen 1 (1:100, Southern Biotech 1310-01), collagen 4 (1:100, Novus NBP1-26549) and fibronectin (1:100; abcam ab2413). Slides were scanned in an automated slide scanner 3DHistech Panoramic 259 Flash III. Analysis was performed with Image J software on an average of 5 images per animal.

Some of the methods involve tissue extracts and acid activation of serum and tissue extracts. Preparation of tissue extract: Isolated solid tissues were rinsed quickly in a sterile normal saline to remove the blood and were briefly placed on a sterile cloth to let dry. To obtain tissue lysates, approximately 30 to 50 mg of tissues were minced and sonicated in 500 μl of lysis buffer (50 mM Tris-HCl pH 7.5) containing 100 mM sodium fluoride, 30 mM sodium pyrophosphate, 2 mM sodium molybdate, 1 mM sodium ortho vanadate, 1 mM glycerophosphate, and 1× protease inhibitor cocktail on ice. Samples were centrifuged at 13,000 rpm for 20 minutes at 4° C. Clear supernatant was collected and stored in aliquots at −70° C. Protein quantification in the lysate was done by the bicinchoninic acid (BCA) method. Acid activation of serum and tissue extracts: To assess the amount of total TGF-β, acid activation was to isolate free TGF-β molecules from latent complex. Briefly, 30 μl of serum or protein lysate (equivalent to 200 μg protein) was added to 100 μl of RIPA, followed by addition of 10 μl of 1 N HCl. Samples were rocked for 1 hour at 4° C. Acid activation was stopped by neutralization with 10 μl of 1 N NaOH. Once acid activated, the samples were stored on ice and were used on the same day.

Those skilled in the art will recognize, or be able to ascertain using no more than routine experimentation, many equivalents to the specific embodiments of the method and compositions described herein. Such equivalents are intended to be encompassed by the following claims.

### Embodiments

Embodiment 1: A method of treating a subject in need thereof comprising applying an alternating electric field, to a target site of the subject in need thereof; and administering a cancer associated fibroblast (CAF) inhibitor to the subject in need thereof.

Embodiment 2: The method of embodiment 1, wherein the CAF inhibitor is a CAF activation inhibitor or a CAF signaling inhibitor.

Embodiment 3: The method of embodiment 2, wherein the CAF activation inhibitor is a fibroblast activation protein (FAP) inhibitor.

Embodiment 4: The method of embodiment 3, wherein the FAP inhibitor is Talabostat, FAP-2286, or TAM558.

Embodiment 5: The method of embodiment 2, wherein the CAF activation inhibitor is a selective PDGFRb inhibitor.

Embodiment 6: The method of embodiment 5, wherein the selective PDFGRb inhibitor is sPDGFRbIg, Crenolanib, Orantinib,CP-673451, SU16f, Tyrphostin AG 1296, Tyrphostin AG1433 or Seralutinib.

Embodiment 7: The method of embodiment 2, wherein the CAF activation inhibitor is a Hsp90 inhibitor.

Embodiment 8: The method of embodiment 7, wherein the Hsp90 inhibitor is XL888, Geldanamycin, Gedunin, Tanespimycin, Luminespib, BIIV 021, CCT 018159, or 17-AAG.

Embodiment 9: The method of embodiment 2, wherein the CAF activation inhibitor is a LRRC15 inhibitor.

Embodiment 10: The method of embodiment 9, wherein the LRRC15 inhibitor is ABBV-085 antibody.

Embodiment 11: The method of embodiment 2, wherein the CAF signaling inhibitor is a Lysyl oxidase (LOX) inhibitor.

Embodiment 12: The method of embodiment 2, wherein the CAF signaling inhibitor is a Lysyl oxidase like (LOXL) inhibitor.

Embodiment 13: A method of reducing or preventing cancer associated fibroblast (CAF) activation comprising applying an alternating electric field, to a population of cells comprising one or more fibroblasts; and contacting a CAF activation inhibitor to the population of cells.

Embodiment 14: A method of reducing cancer associated fibroblast (CAF) signaling comprising: applying alternating electric fields, to a population of cells comprising one or more fibroblasts; and contacting a CAF signaling inhibitor to the population of cells.

Embodiment 15: The method of embodiment 13, wherein the CAF activation inhibitor is a fibroblast activation protein (FAP) inhibitor.

Embodiment 16: The method of embodiment 15, wherein the FAP inhibitor is Talabostat, FAP-2286, or TAM558.

Embodiment 17: The method of embodiment 13, wherein the CAF activation inhibitor is a selective PDGFRb inhibitor.

Embodiment 18: The method of embodiment 17, wherein the selective PDFGRb inhibitor is sPDGFRbIg, Crenolanib, Orantinib, CP-673451, SU16f, Tyrphostin AG 1296, Tyrphostin AG1433 or Seralutinib.

Embodiment 19: The method of embodiment 13, wherein the CAF activation inhibitor is a Hsp90 inhibitor.

Embodiment 20: The method of embodiment 19, wherein the Hsp90 inhibitor is XL888, Geldanamycin, Gedunin, Tanespimycin, Luminespib, BIIV 021, CCT 018159, or 17-AAG.

Embodiment 21: The method of embodiment 13, wherein the CAF activation inhibitor is a LRRC15 inhibitor.

Embodiment 22: The method of embodiment 21, wherein the LRRC15 inhibitor is ABBV-085 antibody.

Embodiment 23: The method of embodiment 14, wherein the CAF signaling inhibitor is a LOX (Lysyl oxidase) inhibitor.

Embodiment 24: The method of embodiment 14, wherein the CAF signaling inhibitor is a LOXL (Lysyl oxidase like) inhibitor.

Embodiment 25: The method of any of the embodiments, wherein the target site comprises one or more cancer cells.

Embodiment 26: The method of any of the embodiments, wherein the alternating electric field is applied before, after, or simultaneously with administering the CAF inhibitor.

Embodiment 27: The method of any of the preceding claims, wherein the CAF inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

Embodiment 28: The method of any one of embodiments 13-27, wherein the population of cells is in vivo.

Embodiment 29: The method of any one of embodiments 13-27, wherein the population of cells is in a subject.

Embodiment 30: The method of any of the embodiments, wherein the CAF activation inhibitor inhibits or decreases fibroblast activation protein (FAP), alpha smooth muscle actin (aSMA), or platelet derived growth factor receptor beta (PDGFRb) expression.

Embodiment 31: The method of any of the embodiments, wherein the CAF activation inhibitor blocks upregulation of FAP, aSMA and/or PDGFRb expression in response to alternating electric fields.

Embodiment 32: The method of any of the embodiments, wherein the CAF activation inhibitor prevents PDGFRb from interacting with or binding to PDGF.

Embodiment 33: The method of any of the embodiments, wherein the CAF activation inhibitor prevents Hsp90 from ATPase activity that upregulates fibroblast activation.

Embodiment 34: The method of any of the embodiments, wherein the CAF inhibitor prevents Lox or Loxl from covalently cross-linking collagen and elastin in extracellular matrix (ECM).

Embodiment 35: The method of any of the embodiments, wherein the alternating electric field has a frequency between 50 kHz and 1 MHz.

Embodiment 36: The method of any of the embodiments, wherein the alternating electric field has a frequency of about 150 or 250 kHz.

Embodiment 37: The method of any of the embodiments, wherein the alternating electric field has a field strength of between 0.5 and 10 V/cm RMS.

Embodiment 38: The method of any of the embodiments, wherein the alternating electric field has a field strength of about 0.9 V/cm RMS.

Embodiment 39: The method of any of the embodiments, further comprising administering a cancer therapeutic.

Embodiment 40: The method of embodiments 1-39, wherein after step a) and prior to step b) detecting an increase in aSMA, FAP, PDGFRP, hedgehog, hyaluronic acid, Hsp90, or CD26 expression in the subject or cell.

Embodiment 41: The method of embodiments 1-40, wherein step b) is performed 1, 2, 3, 4, 5, 6, 7, 8, 9, or 10 days after step a) is performed.

Embodiment 42: A method of decreasing tumor growth in a subject in need thereof comprising a) applying an alternating electric field, to a target site of the subject in need thereof; and b) administering a cancer associated fibroblast (CAF) inhibitor to the subject in need thereof.

Embodiment 43: A method of decreasing tumor invasion in a subject in need thereof comprising: a) applying an alternating electric field, to a target site of the subject in need thereof; and b) administering a cancer associated fibroblast (CAF) inhibitor to the subject in need thereof.

Embodiment 44: A method of decreasing tumor angiogenesis in a subject in need thereof comprising: a) applying an alternating electric field, to a target site of the subject in need thereof; and b) administering a cancer associated fibroblast (CAF) inhibitor to the subject in need thereof.

Embodiment 45: A method of decreasing tumor metastasis in a subject in need thereof comprising: a) applying an alternating electric field, to a target site of the subject in need thereof; and b) administering a cancer associated fibroblast (CAF) inhibitor to the subject in need thereof.

Embodiment 46: The method of any one of embodiments 42-45, wherein the target site comprises cancer cells.

Embodiment 47: The method of any one of embodiments 1-2, 13-14, or 25-46, wherein the CAF activation inhibitor is a selective Hedgehog inhibitor, a selective Hyaluronic acid secretion inhibitor, or an IL-1R inhibitor.

Embodiment 48: The method of any one of embodiments 1-2, 13-14, or 25-46, wherein the CAF signaling inhibitor is a CXCR4 inhibitor or CXCR12 inhibitor.

Embodiment 49: A cancer associated fibroblast inhibitor for use with an alternating electric field in a method of treating a subject in need thereof.

Embodiment 50: An alternating electric field together with a cancer associated fibroblast inhibitor for use in a method of treating a subject in need thereof.

Embodiment 51: The method of embodiments 49-50, wherein the CAF inhibitor is a CAF activation inhibitor or a CAF signaling inhibitor.

Embodiment 52: The method of embodiment 51, wherein the CAF activation inhibitor is a fibroblast activation protein (FAP) inhibitor.

Embodiment 53: The method of embodiment 52, wherein the FAP inhibitor is Talabostat, FAP-2286, or TAM558.

Embodiment 54: The method of embodiment 51, wherein the CAF activation inhibitor is a selective PDGFRb inhibitor.

Embodiment 55: The method of embodiment 54, wherein the selective PDFGRb inhibitor is sPDGFRbIg, Crenolanib, Orantinib,CP-673451, SU16f, Tyrphostin AG 1296, Tyrphostin AG1433 or Seralutinib.

Embodiment 56: The method of embodiment 51, wherein the CAF activation inhibitor is a Hsp90 inhibitor.

Embodiment 57: The method of embodiment 56 wherein the Hsp90 inhibitor is XL888, Geldanamycin, Gedunin, Tanespimycin, Luminespib, BIIV 021, CCT 018159, or 17-AAG.

Embodiment 58: The method of embodiment 51, wherein the CAF activation inhibitor is a LRRC15 inhibitor.

Embodiment 59: The method of embodiment 58, wherein the LRRC15 inhibitor is ABBV-085 antibody.

Embodiment 60: The method of embodiment 51, wherein the CAF signaling inhibitor is a Lysyl oxidase (LOX) inhibitor.

Embodiment 61: The method of embodiment 51, wherein the CAF signaling inhibitor is a Lysyl oxidase like (LOXL) inhibitor.

Embodiment 62: The method of any of the embodiments 49-61, wherein the target site comprises one or more cancer cells.

Embodiment 63: The method of any of the embodiments 49-62, wherein the alternating electric field is applied before, after, or simultaneously with administering the CAF inhibitor.

Embodiment 64: The method of any of the preceding claims 49-63, wherein the CAF inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

Embodiment 65: The method of any of the embodiments 49-64, wherein the CAF activation inhibitor inhibits or decreases fibroblast activation protein (FAP), alpha smooth muscle actin (aSMA), or platelet derived growth factor receptor beta (PDGFRb) expression.

Embodiment 66: The method of any of the embodiments 49-65, wherein the CAF activation inhibitor blocks upregulation of FAP, aSMA and/or PDGFRb expression in response to alternating electric fields.

Embodiment 67: The method of any of the embodiments 49-66, wherein the CAF activation inhibitor prevents PDGFRb from interacting with or binding to PDGF.

Embodiment 68: The method of any of the embodiments 49-67, wherein the CAF activation inhibitor prevents Hsp90 from ATPase activity that upregulates fibroblast activation.

Embodiment 69: The method of any of the embodiments 49-68, wherein the CAF inhibitor prevents Lox or Loxl from covalently cross-linking collagen and elastin in extracellular matrix (ECM).

Embodiment 70: The method of any of the embodiments 49-69, wherein the alternating electric field has a frequency between 50 kHz and 1 MHz.

Embodiment 71: The method of any of the embodiments 49-70, wherein the alternating electric field has a frequency of about 150 or 250 kHz.

Embodiment 72: The method of any of the embodiments, wherein the alternating electric field has a field strength of between 0.5 and 10 V/cm RMS.

Embodiment 73: The method of any of the embodiments 49-72, wherein the alternating electric field has a field strength of about 0.9 V/cm RMS.

Embodiment 74: The method of any of the embodiments 49-73, further comprising administering a cancer therapeutic.

Embodiment 75: The method of embodiments 49-74, wherein after step a) and prior to step b) detecting an increase in aSMA, FAP, PDGFRP, hedgehog, hyaluronic acid, Hsp90, or CD26 expression in the subject or cell.

Embodiment 76: The method of embodiments 49-75, wherein step b) is performed 1, 2, 3, 4, 5, 6, 7, 8, 9, or 10 days after step a) is performed.

We claim:

1. A method of treating a subject in need thereof comprising:
a) applying an alternating electric field, to a target site of the subject in need thereof; and
b) administering a cancer associated fibroblast (CAF) inhibitor to the subject in need thereof.

2. The method of claim 1, wherein the CAF inhibitor is a CAF activation inhibitor or a CAF signaling inhibitor.

3. The method of claim 2, wherein the CAF activation inhibitor is a fibroblast activation protein (FAP) inhibitor, a selective PDGFRb inhibitor, a Hsp90 inhibitor, a LRRC15 inhibitor, a selective Hedgehog inhibitor, a selective Hyaluronic acid secretion inhibitor, or an IL-1R inhibitor.

4. The method of claim 3, wherein the FAP inhibitor is Talabostat, FAP-2286, or TAM558.

5. The method of claim 3, wherein the selective PDFGRb inhibitor is sPDGFRbIg, Crenolanib, Orantinib,CP-673451, SU16f, Tyrphostin AG 1296, Tyrphostin AG1433 or Seralutinib.

6. The method of claim 3, wherein the Hsp90 inhibitor is XL888, Geldanamycin, Gedunin, Tanespimycin, Luminespib, BIIV 021, CCT 018159, or 17-AAG.

7. The method of claim 3, wherein the LRRC15 inhibitor is ABBV-085 antibody.

8. The method of claim 2, wherein the CAF signaling inhibitor is a Lysyl oxidase (LOX) inhibitor, a Lysyl oxidase like (LOXL) inhibitor, CXCR4 inhibitor or CXCR12 inhibitor.

9. A method of reducing or preventing cancer associated fibroblast (CAF) activation comprising:
a) applying an alternating electric field, to a population of cells comprising one or more fibroblasts; and
b) contacting a CAF activation inhibitor to the population of cells.

10. A method of reducing cancer associated fibroblast (CAF) signaling comprising:
a) applying alternating electric fields, to a population of cells comprising one or more fibroblasts; and
c) contacting a CAF signaling inhibitor to the population of cells.

11. The method of claim 1, wherein the target site comprises one or more cancer cells.

12. The method of claim 1, wherein the alternating electric field is applied before, after, or simultaneously with administering the CAF inhibitor.

13. The method of claim 1, wherein the CAF inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

14. The method of claim 2, wherein the CAF activation inhibitor inhibits or decreases fibroblast activation protein (FAP), alpha smooth muscle actin (aSMA), or platelet derived growth factor receptor beta (PDGFRb) expression, and/or
prevents PDGFRb from interacting with or binding to PDGF, and/or
prevents Hsp90 from ATPase activity that upregulates fibroblast activation.

15. The method of claim 1, wherein the CAF inhibitor prevents Lox or Loxl from covalently cross-linking collagen and elastin in extracellular matrix (ECM).

16. The method of claim 1, wherein the alternating electric field has a frequency between 50 kHz and 1 MHz.

17. The method of claim 1, wherein the alternating electric field has a frequency about 150 or 250 kHz.

18. The method of claim 1, wherein the alternating electric field has a field strength of between 0.5 and 10 V/cm RMS.

19. The method of claim 1, wherein the alternating electric field has a field strength of about 0.9 V/cm RMS.
